(19) United States
(12) Patent Application Publication
(10) Pub. No.: US 2026/0242698 A1
(43) Pub. Date: Aug. 20, 2026

(54) COMPOSITIONS CONTAINING BIOSURFACTANTS AND DESFERRIOXAMINES

(71) Applicant: Evonik Operations GmbH, Essen (DE)

(72) Inventors: Kathrin Daniela Brandt, Duesseldorf (DE); Alexander Thiemann, Hasloh (DE); Alexandra Trambitas, Alzenau (DE)

(73) Assignee: Evonik Operations GmbH, Essen (DE)

(21) Appl. No.: 19/139,455

(22) PCT Filed: Dec. 12, 2023
(86) PCT No.: PCT/EP2023/085299

§ 371 Date: Jun. 16, 2025

(30) Foreign Application Priority Data
Dec. 21, 2022 (EP) 22215270.4

(51) Int. Cl. C11D 3/32; A61K 8/42; A61Q 19/10

(52) U.S. Cl. CPC C11D 3/32; A61K 8/42; A61Q 19/10

(57) ABSTRACT

Composition comprising A) at least one substance chosen from desferrioxamines and acid addition products of a desferrioxamine or salts thereof, B) at least one biosurfactant, and optionally C) at least one non-bio surfactant. Use of a composition according to at least one of the preceding claims to remove dirt, preferably fat and/or oil-containing stains from a surface, preferably selected from skin, a dish surface or a textile's surface.

### FIELD OF THE INVENTION

Field of the invention are compositions comprising
A) at least one substance chosen from desferrioxamines and acid addition products of a desferrioxamine or salts thereof,
B) at least one biosurfactant, and optionally
C) at least one non-bio surfactant,
and their use.

### PRIOR ART

Desferrioxamines form a substance category within the iron-binding siderophores. A review is found, for example, in Hider and Kong, Chemistry and Biology of Siderophores, Natural Product Reports (2010), 27(5), 637-657.

Desferrioxamine B, also known as deferoxamine, is described for application in cosmetic applications. Thus, DE10050155 discloses cosmetic and dermatological preparations with a content of desferrioxamine B.

EP2786742 discloses cosmetic formulations containing at least one rhamnolipid.

EP3201302 discloses formulations comprising A) at least one biosurfactant, B) at least one additional surfactant selected from the group of betaines, alkoxylated fatty alcohol sulphates and alkylamine oxides.

EP2787065 discloses detergent formulations for textiles, comprising a mono- and di-rhamnolipid mixture composition, wherein a weight ratio of said di-rhamnolipids to said mono-rhamnolipids is greater than 51:49.

EP3271448 discloses cleaning compositions comprising A) at least one peptidase, B) at least one biosurfactant, and optionally C) at least one anionic surfactant.

It was an objection of the invention to provide for compositions with outstanding properties for use in the cosmetic and in the household care field.

### DESCRIPTION OF THE INVENTION

Surprisingly, it has been found that the compositions described below are able to solve the problem addressed by the invention.

The present invention therefore relates to compositions comprising
A) at least one substance chosen from desferrioxamines and acid addition products of a desferrioxamine or salts thereof, in particular desferrioxamine B,
B) at least one biosurfactant, and optionally
C) at least one non-bio surfactant.

The invention further provides for the use of the inventive compositions to remove dirt, especially fat and/or oil-containing stains.

An advantage of the present invention is that the compositions according to the invention have a particularly low influence on the viscosity of a formulation and can be used over a broad pH range.

An additional advantage of the present invention is that the compositions according to the invention can be incorporated in a particularly easy and stable manner in cosmetic and home care formulations.

Another advantage of the present invention is that the compositions of the instant invention are cold-processable, which results in less energy consumption during processing.

An additional advantage of the present invention is that the compositions according to the invention impart excellent colour stability to cosmetic and home care formulations.

A further advantage of the compositions according to the invention is that they are very mild to the skin.

Another advantage of the present invention is their pleasant odour and taste profile.

Another advantage of the present invention is that the compositions of the instant invention provide effective solubilizing properties for flavours and fragrances.

An additional advantage of the present invention is that the compositions according to the invention increase the microbial stability of cosmetic and homes care formulations.

Another advantage of the present invention is that the compositions according to the invention have superior cleaning power.

A further advantage is that the compositions of the present invention hinder soil redeposition efficiently.

An additional advantage of the present invention is that the compositions according to the invention reduce limescale and increase the gloss of surfaces.

Another advantage of the present invention is that that the compositions of the instant invention support the sheeting effect during the rinsing step, which leads to a fast and residue free drying surfaces.

Another advantage is that the composition of the instant invention exhibits superior properties in terms of unwanted remnants of fragrance and/or odour as well taste on rinsed dishes as well as on skin and hair.

Another advantage of the present invention is that the compositions according to the invention show effective make-up removal properties without dehydrating the skin at the same time.

Another advantage of the present invention is that the compositions of the instant invention provide effective moisturizing properties in cosmetic formulations.

An additional advantage of the present invention is that the compositions according to the invention exhibit particularly good skin sensorics and contribute to a reduced tackiness.

Another advantage of the present invention is that the compositions according to the invention lead to improved colour protection and longer colour retention of dyed or coloured hair.

An additional advantage of the present invention is that the compositions according to the invention contribute to an improved foaming behaviour especially in hard water,

The instant invention encompasses a composition comprising
A) at least one substance chosen from desferrioxamines and acid addition products of a desferrioxamine or salts thereof,
B) at least one biosurfactant, and optionally
C) at least one non-bio surfactant.

Any desferrioxamine and acid addition product of a desferrioxamine or salt thereof, and also mixtures thereof, can be present according to the invention in component A).

Preferred desferrioxamines in this connection are chosen from desferrioxamine A_{1A}, desferrioxamine A_{1B}, desferrioxamine A₂, desferrioxamine B, desferrioxamine D₁, desferrioxamine D₂, desferrioxamine E, desferrioxamine Et₁, desferrioxamine Et₂, desferrioxamine Et₃, desferrioxamine G₁, desferrioxamine G_{2A}, desferrioxamine G_{2B}, desferrioxamine G_{2C}, desferrioxamine H, desferrioxamine N, desferrioxamine P₁, desferrioxamine T₁, desferrioxamine T₂, desferrioxamine T₃, desferrioxamine T₇, desferrioxamine T₈, desferrioxamine Te₁, desferrioxamine Te₂, desferrioxamine Te₃, desferrioxamine X₁, desferrioxamine X₂, desferrioxamine X₃ and desferrioxamine X₄, preferably desferrioxamine B, desferrioxamine D₁, desferrioxamine D₂, desferrioxamine E and desferrioxamine H, particularly preferably desferrioxamine B and desferrioxamine E.

For the nomenclature, compare if necessary, Hider and Kong, Chemistry and Biology of Siderophores, Natural Product Reports (2010), 27(5), 637-657.

It is preferred according to the invention for the acid addition product of the desferrioxamine or salts thereof to be chosen from acid addition products of the acids chosen from formic acid, aminooxyacetic acid, amino acids, anthraquinonecarboxylic acid, succinic acid, aqueous hydrochloric acid, acetic acid, folic acid, glutaric acid, hydroxamic acid, malonic acid, methanesulfonic acid, nalidixic acid, N-(methylamino)benzoic acid, phenylsuccinic acid, phenylglutaric acid, phosphorous acid, phosphoric acid, hydrogen chloride, sulfurous acid, sulfuric acid, tartaric acid and citric acid, hydrogen chloride, methanesulfonic acid, acetic acid and amino acids being particularly preferred.

A preferred composition according to the invention is characterized in that component A) is present in an amount of 0.005% by weight to 10.0% by weight, preferably from 0.05% by weight to 5.0% by weight, particularly preferably from 0.1% by weight to 1.0% by weight, the percentages by weight being with respect to the total composition.

When determining the amount of component A) only the mass of the desferrioxamine is taken into account while disregarding the counter ion of the salt or the part of the acid in the acid addition product, if the case.

Within the context of the present invention, “biosurfactants” are understood as meaning all glycolipids produced by fermentation. The term “biosurfactant” also covers glycolipids that are chemically or enzymatically modified after fermentation, as long as structurally a glycolipid remains. Raw materials for producing the biosurfactants that can be used are carbohydrates, in particular sugars such as e.g. glucose and/or lipophilic carbon sources such as fats, oils, partial glycerides, fatty acids, fatty alcohols, long-chain saturated or unsaturated hydrocarbons.

In the context of the present invention, the terms “surfactant” is understood to mean organic substances having interface-active properties that have the ability to reduce the surface tension of water at 20° C. and at a concentration of 0.5% by weight based on the overall composition to below 45 mN/m. Surface tension is determined by the DuNoüy ring method at 20° C.

Where average values are stated hereinbelow, then, unless stated otherwise, these are number-averaged average values.

Unless stated otherwise, percentages are data in percent by weight.

Wherever measurement values are stated hereinbelow, then, unless stated otherwise, these have been determined at a temperature of 25° C. and a pressure of 1013 mbar.

The “pH” in connection with the present invention—unless stated otherwise—is defined as the value which is measured for the relevant composition at 25° C. after stirring for five minutes using a pH electrode calibrated in accordance with ISO 4316 (1977).

The composition according to the instant invention preferably comprises as component B) at least one biosurfactant selected from rhamnolipids, sophorolipids, glucolipids, cellulose lipids, mannosylerythritol lipids and trehalose lipids, preferably rhamnolipids, sophorolipids and glucolipids, most preferably rhamnolipids. The biosurfactants can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789 or the documents cited therein. Suitable biosurfactants can be acquired e.g. from Soliance, France.

Preferably, the composition according to the instant invention has, as biosurfactant at least one selected from rhamnolipids, in particular mono-, di- or polyrhamnolipids, glucolipids, in particular mono-, di- or polyglucolipids, and sophorolipids, in particular mono-, di- or polysophorolipids, preferably selected from rhamnolipids and glucolipids, most preferably rhamnolipids.

The term “rhamnolipids” in the context of the present invention preferably is understood to mean particularly compounds of the general formula (I) and salts thereof,

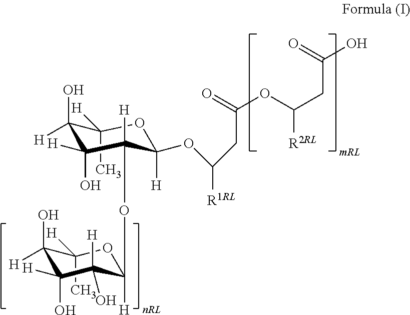

where
mRL=2, 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature:

“diRL-CXCY” are understood to mean di-rhamnolipids of the general formula (I) where nRL=1, in which one of the residues R^{1RL} and R^{2RL}=(CH₂)ₒ—CH₃ where o=X−4 and the remaining residue R¹ or R²=(CH₂)ₒ—CH₃ where o=Y−4.

“monoRL-CXCY” are understood to mean mono-rhamnolipids of the general formula (I), in which one of the residues R^{1RL} and R^{2RL}=(CH₂)ₒ—CH₃ where o=X−4 and the remaining residue R^{1RL} or R^{2RL}=(CH₂)ₒ—CH₃ where o=Y−4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42(6):997-1002.

Rhamnolipids produced by Pseudomonas aeruginosa are commercially available from Jeneil Biotech Inc., e.g. under the tradename Zonix, from Logos Technologies (technology acquired by Stepan), e.g. under the tradename NatSurFact, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The present invention provides a composition preferably comprising as biosurfactant rhamnolipids, characterized in that the biosurfactant component B) comprises
51% by weight to 100% by weight, preferably 60% by weight to 95% by weight, particularly preferably 80% by weight to 90% by weight, of mono-rhamnolipids, especially those of formula (I) with nRL=0,
where the percentages by weight refer to the sum of all of the rhamnolipids present.

This surprisingly elevates the viscosity of the composition according to the instant invention. Preferably this preferred embodiment is combined with a content of soil release agents (see below), preferably of the carboxy methyl inulin type.

The present invention further provides a composition preferably comprising as biosurfactant rhamnolipids, characterized in that the biosurfactant component B) comprises
51% by weight to 95% by weight, preferably 55% by weight to 80% by weight, particularly preferably 60% by weight to 70% by weight, of diRL-C10C10,
where the percentages by weight refer to the sum of all of the rhamnolipids present.

A preferred composition according to the invention is characterized in that the composition comprises as biosurfactant rhamnolipids as described above with a content of
0.5% by weight to 15% by weight, preferably 3% by weight to 12% by weight, particularly preferably 5% by weight to 10% by weight, of diRL-C10C12:1,
where the percentages by weight refer to the sum of all of the rhamnolipids present.

A further preferred composition according to the invention is characterized in that the composition comprises as biosurfactant rhamnolipids as described above with a content of
0.5 to 25% by weight, preferably 3% by weight to 15% by weight, particularly preferably 5% by weight to 12% by weight, of diRL-C10C12,
where the percentages by weight refer to the sum of all of the rhamnolipids present.

A further preferred composition according to the invention is characterized in that the composition comprises as biosurfactant rhamnolipids as described above with a content of
0.1% by weight to 25% by weight, preferably 2% by weight to 10% by weight, particularly preferably 4% by weight to 8% by weight, of diRL-C8C10,
where the percentages by weight refer to the sum total of all rhamnolipids present.

An even further preferred composition according to the invention is characterized in that the composition comprises as biosurfactant rhamnolipids as described above with a content of
0.1% by weight to 5% by weight, preferably 0.5% by weight to 3% by weight, particularly preferably 0.5% by weight to 2% by weight, of monoRL-C8C10 and/or, preferably and
0.1% by weight to 5% by weight, preferably 0.5% by weight to 3% by weight, particularly preferably 0.5% by weight to 2% by weight, of monoRL-C10C10,
where the percentages by weight refer to the sum of all of the rhamnolipids present.

The present invention provides a composition alternatively preferably comprising as biosurfactant rhamnolipids, characterized in that the biosurfactant component B) comprises
10% by weight to 50% by weight, preferably 20% by weight to 40% by weight, particularly preferably 25% by weight to 35% by weight, of monoRL-C10C10 and
where the percentages by weight refer to the sum of all of the rhamnolipids present.

The alternatively preferred composition according to the invention is preferably characterized in that the composition comprises as biosurfactant rhamnolipids as described above with a content of 10% by weight to 30% by weight, preferably 12% by weight to 25% by weight, particularly preferably 15% by weight to 20% by weight, of diRL-C10C10,
where the percentages by weight refer to the sum of all of the rhamnolipids present.

The alternatively preferred composition according to the invention is characterized in that the composition comprises as biosurfactant rhamnolipids as described above with a content of
10% by weight to 30% by weight, preferably 12% by weight to 25% by weight, particularly preferably 15% by weight to 20% by weight, of monoRL-C8C10,
where the percentages by weight refer to the sum of all of the rhamnolipids present.

The alternatively preferred composition according to the invention is characterized in that the composition comprises as biosurfactant rhamnolipids as described above with a content of
3% by weight to 25% by weight, preferably 5% by weight to 20% by weight, particularly preferably 10% by weight to 15% by weight, of monoRL-C10C12:1,
where the percentages by weight refer to the sum total of all rhamnolipids present.

The alternatively preferred composition according to the invention is characterized in that the composition comprises as biosurfactant rhamnolipids as described above with a content of
1% by weight to 15% by weight, preferably 2% by weight to 10% by weight, particularly preferably 3% by weight to 8% by weight, of diRL-C10C12,
where the percentages by weight refer to the sum of all of the rhamnolipids present.

In the context of the present invention, the term “sophorolipids” preferably is understood as meaning compounds of the general formulae (IIa) and (IIb) and salts thereof

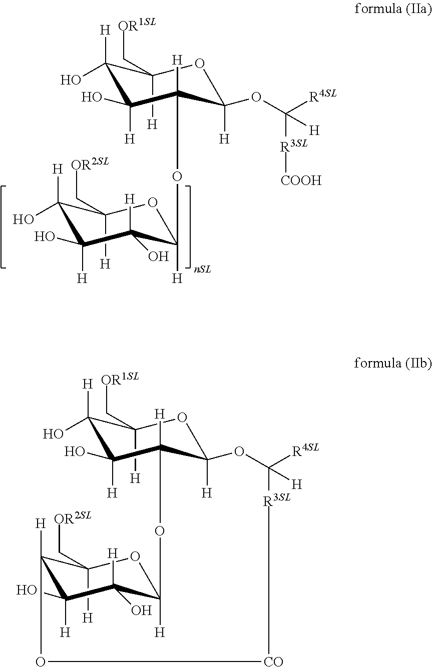

where
R^{1SL}=H or CO—CH₃,
R^{2SL}=H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}=H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be used in accordance with the invention in their acid form or their lactone form. Preferred compositions according to the instant invention comprise a sophorolipid in which the ratio by weight of lactone form to acid form is in the range of 20:80 to 80:20, especially preferably in the ranges of 30:70 to 40:60.

To determine the content of sophorolipids in the acid or lactone form in a formulation, refer to EP1411111B1, page 8, paragraph [0053].

In connection with the present invention, the term “glucolipids” preferably is understood as meaning compounds of the general formula (III) and salts thereof,

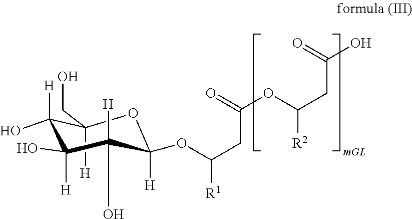

where
mGL=3, 2, 1 or 0, preferably 1 or 0,
R^{1GL} and R^{2GL}=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.

Distinct glucolipids are abbreviated according to the following nomenclature:
“GL-CXCY” is understood as meaning glucolipids of the general formula (III) in which one of the radicals R^{1GL} and R^{2GL}=(CH₂)ₒ—CH₃ where o=X−4 and the remaining radical R^{1GL} or R^{2GL}=(CH₂)ₒ—CH₃ where o=Y−4.

The nomenclature used thus does not differentiate between “CXCY” and “CYCX”.

If one of the aforementioned indices X and/or Y is provided with “:Z”, then this means that the respective radical R^{1GL} and/or R^{2GL}=an unbranched, unsubstituted hydrocarbon radical with X−3 or Y−3 carbon atoms having Z double bonds.

Methods for production of glucolipids can be carried out as described in WO2019154970.

The compositions according to the instant invention can bear high concentrations of the components to be advantageously useful, e.g. in the production of end customer formulations.

In this context it is preferred in the instant invention, that the composition according to the instant invention is characterized in that it contains component A) in an amount from 0.1% by weight to 75.0% by weight, preferably from 0.5% by weight to 55.0% by weight, particularly preferably from 1.0% by weight to 40% by weight, based on the total composition.

It is preferred in the instant invention, that the composition according to the instant invention is characterized in that it contains component B) in an amount from 5.0% by weight to 80% by weight, preferably from 10.0% by weight to 75% by weight, particularly preferably from 25% by weight to 60% by weight, based on the total composition.

The composition according to the instant invention preferably comprises at least one non-bio surfactant as component C).

It is preferred in the instant invention, that the composition according to the instant invention is characterized in that it contains component C) in an amount from 1.0% by weight to 50% by weight, preferably from 5.0% by weight to 30% by weight, particularly preferably from 5.0% by weight to 20% by weight, based on the total composition.

Preferably the non-bio surfactant contained in the composition according to the instant invention is selected from anionic surfactants, preferably selected from the group comprising, preferably consisting of, anionic, cationic, non-ionic, semi-polar and zwitterionic surfactants, more preferably anionic surfactants.

Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS) and sodium lauryl sulfate (SLS), fatty alcohol sulfates (FAS), primary alcohol sulfates (PAS), alkyl ethersulfates (AES or AEOS or FES, also known as alcohol ethoxysulfates or fatty alcohol ether sulfates) such as sodium laureth sulfate (SLES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkoyl isethionates, alkoyl sarcosinates, alkyl sulfoacetates, alkyl sulfosuccinate, alkyl- or alkenylsuccinic acid, dodecenyl/tetradecenyl succinic acid (DTSA), fatty acid derivatives of amino acids (AAS), diesters and monoesters of sulfo-succinic acid or soap, and combinations thereof.

Non-limiting examples of cationic surfactants include alklydimethylethanolamine quat (ADMEAQ), cetyltrimethylammonium bromide (CTAB), behenyltrimethylammonium bromide (BTAB), dimethyldistearylammonium chloride (DSDMAC), didecyldimethylammonium chloride (DDAC), alkylbenzyldimethylammonium chloride, distearoylethyldimonium chloride, bis-(isostearoyl/oleyl isopropyl)dimonium methosulfate, undecylenamidopropyltrimonium methosulfate, alkyl quaternary ammonium compounds, alkoxylated quaternary ammonium (AQA) compounds, and combinations thereof.

Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), polyglycerol esters, glycerol esters, propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

Non-limiting examples of zwitterionic surfactants include betaines, alkyldimethylbetaines, sulfobetaines/sultaines, hydroxysultaines, amphoacetates, amphodiacetates, cocoamphopropionates and combinations thereof.

The composition according to the instant invention preferably is characterized in that the total sum of all surfactants is from 5.0% by weight to 90% by weight, preferably 10.0% by weight to 80% by weight, preferably 20% by weight to 75% by weight, wherein the percentages by weight relate to the total composition.

It is preferred in the instant invention, that the composition according to the instant invention is characterized in that the sum of all biosurfactant in the total surfactant is from 1% by weight to 90% by weight, preferably from 2% by weight to 80% by weight, particularly preferably from 51% by weight to 60% by weight, based on the total amount of surfactant in the composition.

The composition according to the instant invention preferably comprises
D) water.

The composition according to the instant invention preferably has a pH of 4.0 to 12.5, preferably of 5.0 to 10.0, particularly preferably of 5.5 to 9.0.

If the composition according to the instant invention is used, for example, in laundry detergents, it preferably has a pH of 7.0 to 12.5, preferably of 7.5 to 12.0, particularly preferably of 8.0 to 12.0.

If the composition according to the instant invention is used, for example, in manual dishwashing or cosmetic agents, it preferably alternatively has a pH of 4 to 8, preferably of 4.5 to 7.5, particularly preferably of 5 to 6.5.

The composition according to the instant invention can be formulated as a household care composition; preferably in this instance the following applies:

The composition according to the instant invention can further comprise one or more auxiliary agents selected from the group consisting of bleaching systems, hydrotropes, polymers, which may be synthetic, biopolymers, anti-redeposition aids, fiber protection agents, soil release agents, dye transfer inhibitors, fabric hueing agents, opacifiers, blueing dyes, enzyme stabilizing agents, solvents, viscosity modifiers, preservatives, pH-regulators and salts like NaCl and Na2SO4.

Suitable non-aqueous solvents include monohydric or polyhydric alcohols, alkanolamines or glycol ethers, provided they are miscible with water in the specified concentration range.

The solvents are preferably selected from ethanol, n-propanol, i-propanol, butanols, glycol, propanediol, butanediol, glycerine, diglycol, propyldiglycol, butyldiglycol, hexylene glycol, ethylene glycol methyl ether, ethylene glycol ethyl ether, ethylene glycol propyl ether, n-butyl glycol ether, ethylene glycol mono-glycol ether, Diethylene glycol ethyl ether, propylene glycol methyl ether, propylene glycol ethyl ether, Propylene glycol propyl ether, dipropylene glycol monomethyl ether, dipropylene glycol monoethyl ether, diisopropylene glycol monomethyl ether, diisopropylene glycol monoethyl ether, methoxy triglycol, ethoxy triglycol, butoxy triglycol, 1-butoxyethoxy-2-propanol, n-butoxyethoxy-2-propanol, di-butoxyethoxy-2-propanol, 3-butyl-3-methoxyether solvents, and mixtures of these solvents, butoxyethoxy-2-propanol, 3-butyl-3-butyl ether, propyl glycol, di-octanol ether, di-butoxy-2-propanol, 3-butyl-3-butylether, propanol, propylenglycol, di-butoxyethoxy-2-propanol, di-butoxyethoxy-2-propanol, 3-butoxy-3-methoxyether solvents, diisopropylene glycol monomethylether, diisopropylene glycol monomethylether, and mixtures of these solvents. However, it is preferred that the surfactant preparation contains a polyol as the non-aqueous solvent. The polyol can comprise glycerol, 1,2-propanediol, 1,3-propanediol, ethylene glycol, diethylene glycol and/or dipropylene glycol.

Any preservative known in the art for use in detergents may be utilized. Exemplary preservatives include phenoxyethanol, sodium levulinate, sodium benzoate, p-anisic acid, potassium sorbate, benzoic acid, glyceryl caprylate, capryl glycol, penthylene glycol, methyl propane diol, bronopol, isothiazolinone (methylisothiazolinone, chloromethylisothiazolinone) and combinations thereof

Preferably the composition according to the instant invention comprises one or more encapsulate comprising a benefit agent, preferably a sensorial benefit agent. Preferred encapsulates in this context comprise shear/pressure-sensitive action encapsulates, whereby the sensorial benefit agent is released in response to mechanical force (e.g., friction, pressure, shear stress) on the encapsulate. The encapsulate shell is preferably comprised of materials including but not limited to polyurethane, polyamide, polyolefin, polysaccharide, protein, silicone, lipid, modified cellulose, gums, polyacrylate, polyphosphate, polystyrene, polyesters or combinations of these materials. Preferably the sensorial benefit agent comprises a skin benefit agent or an olfactory benefit agent and/or may be a volatile benefit agent. Sensorial benefit agents may also have benefits for hair and/or hard surfaces and/or fabrics. The sensorial benefit may have anti-foam properties, and as such it is advantageous for foaming purposes that it is encapsulated so as not interfering with the foam until release by rubbing. Suitable volatile benefit agents include but are not limited to perfumes, insect repellents, essential oils, sensates such as menthol and aromatherapy actives, preferably perfumes. Mixtures of volatile benefit agents may be used. The total amount of benefit agent is preferably from 0.01 to 10% by weight, more preferably from 0.05 to 5% by weight, even more preferably from 0.1 to 4.0%, most preferably from 0.15 to 4.0% by weight, based on the total weight of the composition. The preferred benefit agent is a perfume. The composition of the instant invention may also comprise an unconfined (also called non-encapsulated) volatile benefit agent. Where the volatile benefit agent is a perfume, the perfumes described below are suitable for use as the encapsulated volatile benefit agent and also as the unconfined perfume component.

The composition according to the instant invention can be formulated as a cosmetic composition; preferably in this instance the following applies:

The compositions according to the invention can further comprise at least one additional component selected from the group of
emollients,
emulsifiers,
thickeners/viscosity regulators/stabilizers,
UV light protection filters,
antioxidants,
hydrotropes,
solids and fillers,
film formers,
pearlescence additives,
deodorant and antiperspirant active ingredients,
insect repellents,
self-tanning agents,
preservatives,
conditioning agents,
perfumes,
dyes,
odour absorbers,
superfatting agents,
other solvents.

Substances which can be used as exemplary representatives of the individual groups are known to those skilled in the art and can be found for example in German application DE 102008001788.4. This patent application is hereby incorporated as reference and thus forms part of the disclosure. As regards further optional components and the amounts used of these components, reference is made expressly to the relevant handbooks known to those skilled in the art, for example K. Schrader, “Grundlagen und Rezepturen der Kosmetika [Cosmetics—fundamentals and formulations]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

The compositions according to the instant invention can be used in different fields of applications, for example in various cleaning applications.

In this context preferred composition according to the instant inventions are formulations, preferably cosmetic, laundry or dishwashing formulations.

It is preferred in the instant invention, that the formulation according to the instant invention is characterized in that it contains component A) in an amount from 0.005% by weight to 10.0% by weight, preferably from 0.01% by weight to 7.0% by weight, particularly preferably from 0.05% by weight to 2.0% by weight, based on the total composition.

It is preferred in the instant invention, that the formulation according to the instant invention is characterized in that it contains component B) in an amount from 0.05% by weight to 25.0% by weight, preferably from 0.1% by weight to 20.0% by weight, particularly preferably from 0.5% by weight to 15.0% by weight, based on the total composition.

The composition according to the instant invention is outstanding in its ability to remove dirt, especially oily dirt, from a surface.

Therefore, a further aspect of the instant invention is the use of a composition according to the instant invention to remove dirt, preferably fat and/or oil-containing stains from a surface, preferably selected from skin, a dish surface or a textile's surface.

In the case of the use according to the invention, particularly preferred embodiments of the compositions according to the instant invention and preferred components thereof are used in the preferred amounts.

The examples adduced hereinafter describe the present invention by way of example, without any intention that the invention, the scope of application of which is apparent from the entirety of the description and the claims, be restricted to the embodiments specified in the examples.

### EXAMPLES

### Example 1: Removal of Unpleasant Odors on Skin

Rhamnolipids were prepared as described in example 1 of EP3023431.

The sophorolipid used is a sophorolipid REWOFERM® SL ONE from Evonik, which has a lactone to acid ratio of 40:60.

Glucolipids were produced according to example 2 of WO2019154970 via fermentation.

The given weight percentages in this example refer to the amount of biosurfactant in the total composition.

An unpleasant odor on the skin of the hands after processing leek-like foods, including onions and garlic, is a much described and widely known problem.

Usually, the harsh onion stink clings to the skin even after thorough washing. It has been found that the compositions according to the invention are able to solve the problem to get rid of that smell.

### Olfactory and Sensory Hand Wash Test

The following example demonstrates the superior odor reduction and improved skin feel of formulations containing compositions according to the present invention.

The test formulation described in table 1 have been prepared according to the following protocol: Initially, the ingredients of phase A are mixed one after the other under medium stirring. Subsequently, phase B is pre-mixed and heated up to 60° C. until all components are completely dissolved. Phase B is then added to phase A under stirring. Phase C is added, and the pH value is adjusted with phase D.

According to the above-described method, five test formulations were prepared (table 1):
formulation I, that was subsequently used as a reference formulation.
formulation II, II b and II c, that contain ingredients of reference formulation I and a biosurfactant.
formulation III, III b and III c, that contain ingredients of the reference formulation I and a composition according to the present invention.

TABLE 1
Hand soap formulations for olfactory and sensory hand wash test
Input concentrations given in wt. % of active ingredient; pH 5.4

| | | Formulations | | | | | | |
|---|---|---|---|---|---|---|---|---|
| Phase | Ingredient | I | II | II b | II c | III¹⁾ | III b¹⁾ | III c¹⁾ |
| A | Rhamnolipid | — | 2.5 | — | — | 2.5 | — | — |
| | Sophorolipid | — | — | 3.0 | — | — | 3.0 | — |
| | Glucolipid | — | — | — | 2.5 | — | — | 2.5 |
| | Coco-Glucoside | 10.0 | 10.0 | 10.0 | 10.0 | 10.0 | 10.0 | 10.0 |
| | (Plantacare 818 UP, BASF) | | | | | | | |
| | Lauryl Glucoside (Plantacare | 5.0 | 5.0 | 5.0 | 5.0 | 5.0 | 5.0 | 5.0 |
| | 1200 UP, BASF) | | | | | | | |
| | PCA Glyceryl Oleate (dermofeel | 1.0 | 1.0 | 1.0 | 1.0 | 1.0 | 1.0 | 1.0 |
| | P-30 MB, Evonik) | | | | | | | |
| | Sorbitan Caprylate; Glyceryl | 0.25 | 0.25 | 0.25 | 0.25 | 0.25 | 0.25 | 0.25 |
| | Oleate | | | | | | | |
| | (TEGO ® Remo 95 MB, Evonik) | | | | | | | |
| | Isoamyl Laurate | 0.5 | 0.5 | 0.5 | 0.5 | 0.5 | 0.5 | 0.5 |
| | (dermofeel sensolv MB, Evonik) | | | | | | | |
| B | Aqua | ad 100 | ad 100 | ad 100 | ad 100 | ad 100 | ad 100 | ad 100 |
| | Sodium Coco-Sulfate | 5.0 | 5.0 | 5.0 | 5.0 | 5.0 | 5.0 | 5.0 |
| | (Sulfopon 1216 G MB, BASF) | | | | | | | |
| | Glycerin; Aqua; Sodium | 3.5 | 3.5 | 3.5 | 3.5 | 3.5 | 3.5 | 3.5 |
| | Levulinate; Sodium Anisate | | | | | | | |
| | (dermosoft 1388 eco, Evonik) | | | | | | | |
| | Glycerin | 7.2 | 7.2 | 7.2 | 7.2 | 7.2 | 7.2 | 7.2 |
| | (Glycerin 99.5% Ph. Eur., | | | | | | | |
| | Cremer Oleo) | | | | | | | |
| C | Desferrioxamine | — | — | — | — | 0.1 | 0.5 | 0.5 |
| D | Citric Acid (sol. 20%) | Ad pH | Ad pH | Ad pH | Ad pH | Ad pH | Ad pH | Ad pH |

¹⁾Formulation according to the invention

Subsequently, the odor reduction and skin feel of the test formulations was evaluated in an olfactory and sensory hand wash test that was performed according to the following protocol:

The test was performed with the help of a trained test panel consisting of 7 test persons. The hands of the test subjects were prewashed with an ethanolic solution for 20 sec. Subsequently, both palms of the subjects are treated with the cut side of half a vegetable onion, one after the other for 20 sec each. The skin is then allowed to dry for one minute. The hands of the test subjects are then washed with 2 ml of the test formulations according to a standardized test protocol. The smell and the feel of the skin are assessed immediately after washing on a scale of 1 (very bad skin feel resp. very strong smell) to 5 (very good skin feel resp. no smell)

TABLE 2
Results of the olfactory and sensory hand wash test; mean value over all test subjects

| | Form. | Form. | Form. | Form. | Form. | Form. | Form. |
|---|---|---|---|---|---|---|---|
| | I | II | II b | II c | III | III b | III c |
| Onion smell | 1.9 | 2.8 | 2.4 | 2.4 | 3.1 | 2.9 | 3.4 |
| (1 = very strong smell to 5 = no smell) | | | | | | | |
| Skin softness | 2.6 | 2.8 | 2.9 | 3.1 | 3.7 | 3.6 | 4.0 |
| (1 = low to 5 = high) | | | | | | | |

Surprisingly, it was found that the formulations according to the invention were better able to remove the onion odor from the skin than the formulations not according to the invention. Furthermore, the formulations according to the invention have resulted into an improved skin softness compared to the formulations not according to the invention.

### Further Formulation Examples

The following examples are for the purpose of illustration and are not to be construed as limitations of the present invention since many variations thereof are possible without departing from its scope.

The example formulations are made using conventional methods. If necessary, the pH value is adjusted by addition of either aqueous sodium hydroxide or citric acid at the end of the manufacturing process. If the preparation of the formulation requires the separate preparation or mixing of formulation ingredients beforehand, this is referred to as a multiphase preparation. If two-phase preparation is required, the two phases are designated accordingly in the tables given below.

The raw materials used are listed with their INCI names. Unless otherwise stated, the data in the tables are given in wt. %.

For recipes 1-27, Rhamnolipid refers to RHEANCE® One, while in recipes 28-106, Rhamnolipid REWOFERM® RL 100 was used.

The sophorolipid used is a sophorolipid REWOFERM® SL ONE from Evonik, which has a lactone to acid ratio of 40:60.

Glucolipids were produced according to example 2 of WO2019154970 via fermentation.

The given weight percentages in the following examples refer to the amount of biosurfactant.

Recipes 1a, 1b, 1c: Shower Cream

| Ingredients [wt. %] | 1a | 1b | 1c |
|---|---|---|---|
| Water | ad 100.0 | ad 100.0 | ad 100.0 |
| Sodium Laureth Sulfate (Texapon NSO, BASF, 28%) | 25.0 | 25.0 | 25.0 |
| Coco-Glucoside (Plantacare 818 UP, BASF, 51%) | 8.0 | 8.0 | 8.0 |
| Cocamidopropyl Betaine (TEGO ® Betain F 50, Evonik, 38%) | 8.0 | 8.0 | 8.0 |
| Rhamnolipid | 0.74 | — | — |
| Sophorolipid | — | 1.0 | — |
| Glucolipid | — | — | 1.0 |
| PEG-18 Glyceryl Oleate/Cocoate (ANTIL ® 171, Evonik) | 1.5 | 1.5 | 1.5 |
| Sorbitan Sesquicaprylate (ANTIL ® Soft SC, Evonik) | 0.8 | 0.8 | 0.8 |
| Glyceryl Oleate (TEGIN ® O V, Evonik) | 0.8 | 0.8 | 0.8 |
| Perfume Spicy Herbs (IFF) | 0.2 | 0.2 | 0.2 |
| Polyglyceryl-4 Caprate (TEGOSOFT ® PC 41, Evonik) | 0.6 | 0.6 | 0.6 |
| Helianthus Annuus Seed Oil (AEC Sunflower Oil, A & E Connock, | 0.2 | 0.2 | 0.2 |
| Perfumery & Cosmetics Ltd.) | | | |
| Linalool (Lipofresh, Lipo Chemicals, Inc.) | 0.1 | 0.1 | 0.1 |
| Coumarin (Rhodiascent extra pure, Solvay Rhodia) | 0.1 | 0.1 | 0.1 |
| Glycerin (Glycerol EP, vegetable, Spiga Nord) | 0.4 | 0.4 | 0.4 |
| Hydroxypropyl Methylcellulose (TEGOCEL ® HPM 50, Evonik) | 0.2 | 0.2 | 0.2 |
| Glycol Distearate (TEGIN ® G 1100 Pellets, Evonik) | 0.4 | 0.4 | 0.4 |
| Sodium Chloride | 0.5 | 0.5 | 0.5 |
| Hydroxypropyl Guar Hydroxypropyltrimonium Chloride (Jaguar C- | 0.2 | 0.2 | 0.2 |
| 162, Solvay Rhodia) | | | |
| dermofeel TOCO 70 non GMO | 0.1 | 0.1 | 0.1 |
| Desferrioxamine | 0.1 | 0.1 | 0.1 |
| Preservative | q.s. | q.s. | q.s. |
| Citric Acid | ad pH 5.2 | ad pH 5.2 | ad pH 5.2 |

Recipes 2a, 2b: Shower Gel

| | Ingredients [wt. %] | 2a | 2b |
|---|---|---|---|
| Phase A | Aqua (Tap Water) | Ad 100 | Ad 100 |
| | Desferrioxamine | 0.1 | 0.1 |
| | Aqua; Sodium Levulinate; Potassium Sorbate | 2.0 | 2.0 |
| | (Verstatil ® SL non GMO; Evonik) | | |
| Phase B | Polyglyceryl-10 Laurate; Aqua; Citric Acid | 3.15 | 3.15 |
| | (dermofeel ® G 10 LW 70 MB) | | |
| | Rhamnolipid | 3.0 | |
| | Sophorolipid | | 3.55 |
| | PCA Glyceryl Oleate (dermofeel ® P-30 | 0.5 | 0.5 |
| | MB; Evonik) | | |
| | Sodium Coco-Sulfate (Sulfopon 1216 G; | 4.0 | 4.0 |
| | BASF) | | |
| | Aqua; Coco-Glucoside; Glyceryl Oleate; | 1.0 | 1.0 |
| | Citric Acid; Tocopherol; Hydrogenated Palm | | |
| | Glycerides Citrate (Lamesoft PO 65; BASF) | | |
| Phase C | Desferrioxamine | 0.1 | 0.1 |
| | Perfume | 0.1 | 0.1 |
| Phase D | Citric Acid Monohydrate | 0.1 | 0.1 |
| | Sodium Chloride | 0.1 | 0.1 |

Recipes 2a, 2b, 2c: Body Shampoo

| | Ingredients [wt. %] | 2a | 2b | 2c |
|---|---|---|---|---|
| Phase | Rhamnolipid | 2.5 | | 1.25 |
| A | Sophorolipid | | 2.5 | 1.25 |
| | Lavandula Angustifolia (Lavender) Oil (AEC | 0.2 | 0.2 | 0.2 |
| | Lavender Oil, A&E Connock Ltd.) | | | |
| | Perfume | 0.1 | 0.1 | 0.1 |
| Phase | Sodium Cocoamphoacetate (REWOTERIC ® AM C, | 10.0 | 10.0 | 10.0 |
| B | Evonik, 32%) | | | |
| Phase | Water | ad 100.0 | ad 100.0 | ad 100.0 |
| C | Xanthan Gum (Keltrol CG-SFT, CP Kelco) | 1.2 | 1.2 | 1.2 |
| Phase | Sodium Lauroyl Methyl Isethionate (Iselux, | 4.5 | 4.5 | 4.5 |
| D | Innospec Active Chemicals) | | | |
| | Capryl/Capramidopropyl Betaine (TEGO ® Betaine | 4.5 | 4.5 | 4.5 |
| | 810, Evonik, 38%) | | | |
| | Citric Acid | 1.2 | 1.2 | 1.2 |
| Phase | Water | 10.0 | 10.0 | 10.0 |
| E | Polyquaternium-7 (Merquat 550, Nalco) | 0.4 | 0.4 | 0.4 |
| | Desferrioxamine | 0.2 | 0.2 | 0.2 |
| | Preservative | q.s. | q.s. | q.s. |

Recipes 3a, 3b, 3c: Shampoo

| | Ingredients [wt. %] | 3a | 3b | 3c |
|---|---|---|---|---|
| Phase | Rhamnolipid | 2.0 | | |
| A | Sophorolipid | | 2.5 | |
| | Glucolipid | | | 2.5 |
| | Isopropyl Myristate (TEGOSOFT ® M, Evonik) | 0.2 | 0.2 | 0.2 |
| | Perfume | 0.1 | 0.1 | 0.1 |
| Phase | Water | ad 100.0 | ad 100.0 | ad 100.0 |
| B | | | | |
| Phase | Sodium Lauryl Sulfate (Texapon LS 35, BASF, | 28.0 | 28.0 | 28.0 |
| C | 30%) | | | |
| Phase | Cocamidopropyl Betaine (TEGO ® Betain F 50, | 9.0 | 9.0 | 9.0 |
| D | Evonik, 38%) | | | |
| Phase | Cocamide MEA (REWOMID ® C 212, Evonik) | 2.0 | 2.0 | 2.0 |
| E | Xanthan Gum (Keltrol CG-SFT, CP Kelco) | 0.3 | 0.3 | 0.3 |
| | Water | 10.0 | 10.0 | 10.0 |
| Phase | Water | 10.0 | 10.0 | 10.0 |
| F | Polyquaternium-10 (Polymer JR 400, Amerchol) | 0.2 | 0.2 | 0.2 |
| Phase | Citric Acid | ad | ad | ad |
| G | | pH 5.0 | pH 5.0 | pH 5.0 |
| | Desferrioxamine | 0.05 | 0.05 | 0.05 |
| Phase | Preservative | q.s. | q.s. | q.s. |
| H | | | | |

Recipes 4a, 4b, 4c: Shampoo

| Ingredients [wt. %] | 4a | 4b | 4c |
|---|---|---|---|
| Water | ad 100.0 | ad 100.0 | ad 100.0 |
| Cocamidopropyl Betaine (TEGO ® Betain F 50, Evonik, | 22.0 | 22.0 | 22.0 |
| 38%) | | | |
| Rhamnolipid | 3.0 | | 2.0 |
| Sophorolipid | | 3.55 | 2.4 |
| Sodium Cocoyl Glutamate (Plantapon ACG HC, BASF) | 1.5 | 1.5 | 1.5 |
| Sodium Cocoyl Glycinate (Hostapon SG, Clariant) | 0.8 | 0.8 | 0.8 |
| Zinc Pyrithione (Microcare ZP, Thor) | 0.1 | 0.1 | 0.1 |
| PEG-120 Methyl Glucose Dioleate (ANTIL ® 120 Plus, | 0.4 | 0.4 | 0.4 |
| Evonik) | | | |
| Sodium Chloride | 0.5 | 0.5 | 0.5 |
| Isostearamide MIPA; Glyceryl Laurate (ANTIL ® SPA 80, | 0.5 | 0.5 | 0.5 |
| Evonik) | | | |
| Xanthan Gum (Keltrol CG-SFT, CP Kelco) | 0.3 | 0.3 | 0.3 |
| Hydroxypropyl Guar Hydroxypropyltrimonium Chloride | 0.3 | 0.3 | 0.3 |
| (Jaguar C-162, Solvay Rhodia) | | | |
| Quaternium-80 (ABIL ® Quat 3272, Evonik) | 0.4 | 0.4 | 0.4 |
| Palmitamidopropyltrimonium Chloride (VARISOFT ® PATC, | 0.4 | 0.4 | 0.4 |
| Evonik) | | | |
| Argania Spinosa Oil (Argan Oil, DSM Nutritional Products | 0.1 | 0.1 | 0.1 |
| Ltd.) | | | |
| Glycerin (Glycerol EP, vegetable, Spiga Nord) | 0.6 | 0.6 | 0.6 |
| Tetrasodium EDTA (Versene 100, The Dow Chemical | 0.1 | 0.1 | 0.1 |
| Company) | | | |
| Caffeine (Merck KGaA /EMD Chemicals, Inc.) | 0.1 | 0.1 | 0.1 |
| Hydrolyzed Wheat Protein (Gluadin WLM, BASF) | 0.1 | 0.1 | 0.1 |
| Limonene (Dipentene No. 122, Hercules Inc.) | 0.1 | 0.1 | 0.1 |
| Citric Acid | ad pH 5.5 | ad pH 5.5 | ad pH 5.5 |
| Sodium Phytate; Aqua; Alcohol (dermofeel ® PA-3; Evonik | | 0.1 | 0.1 |
| Dr. Straetmans GmbH (PA-3) | | | |
| Perfume | 0.2 | 0.2 | 0.2 |
| Desferrioxamine | 0.5 | 0.5 | 0.5 |
| Aqua; Sodium Levulinate; Sodium Benzoate (Verstatil ® BL | | 1.2 | 1.2 |
| non GMO; Evonik Dr. Straetmans GmbH) | | | |

Recipes 5a, 5b, 5c: Liquid Soap

| Ingredients [wt. %] | 5a | 5b | 5c |
|---|---|---|---|
| Water | ad 100 | ad 100 | ad 100 |
| Glycerin (Glycerol EP, vegetable, Spiga Nord) | 4.0 | 4.0 | 4.0 |
| Alcohol | 4.0 | 4.0 | 4.0 |
| Sodium Coco-Sulfate (Texapon HC G, BASF) | 3.0 | 3.0 | 3.0 |
| Rhamnolipid | 1.5 | | |
| Sophorolipid | | 2.0 | |
| Glucolipid | | | 1.5.0 |
| Xanthan Gum (Keltrol CG-SFT, CP Kelco) | 1.5 | 1.5 | 1.5 |
| Mangifera Indica (Mango) Fruit Extract (Mango Extract, Draco | 0.5 | 0.5 | 0.5 |
| Natural Products) | | | |
| Limonene (Dipentene No. 122, Hercules Inc.) | 0.1 | 0.1 | 0.1 |
| Linalool (Lipofresh, Lipo Chemicals, Inc.) | 0.1 | 0.1 | 0.1 |
| Citric Acid | ad | ad | ad |
| | pH 4.9 | pH 4.9 | pH 4.9 |
| Preservative | q.s. | q.s. | q.s. |
| Desferrioxamine | 1.0 | 1.0 | 1.0 |
| Dyes | q.s. | q.s. | q.s. |

Recipes 6a, 6b: Liquid Soap

| | Ingredients [wt. %] | 6a | 6b |
|---|---|---|---|
| Phase | Zinc Ricinoleate; Tetrahydroxyproyl Ethylenediamine; Laureth-3; | 0.80 | 0.80 |
| A | Propylene Glycol (TEGODEO ® CW 90 E MB, Evonik) | | |
| | Polyglyceryl-3 Caprylate (TEGO ® Cosmo P 813 MB, Evonik) | 0.50 | 0.50 |
| | Aqua; Sodium Laureth Sulfate (Emal 228HP, Kao) | 26.00 | 26.00 |
| | Perfume | 0.30 | 0.30 |
| | Water, deionized | Ad 100 | Ad 100 |
| | Cocamidopropyl Betaine (TEGO ® Betain F 50, Evonik) | 8.50 | 8.50 |
| | Lauryl Glucoside, 50% (Plantacare 1200 UP, BASF) | 3.25 | 3.25 |
| | Aqua; Sodium Levulinate; Potassium Sorbate (Verstatil ® SL; Evonik) | 2.00 | 2.00 |
| | Rhamnolipids | 0.25 | |
| | Sophorolipids | | 0.25 |
| | Desferrioxamin | 0.10 | 0.10 |
| Phase | Sodium Chloride | | |
| B | | | |

Recipes 7a, 7b, 7c: Cream Soap

| Ingredients [wt. %] | 7a | 7b | 7c |
|---|---|---|---|
| Water | ad 100 | ad 100 | ad 100 |
| Propylene Glycol (Euxyl K 320, Schülke & Mayr GmbH) | 2.0 | 2.0 | 2.0 |
| Coco-Glucoside (Plantacare 818 UP, BASF, 51) | 10.0 | 10.0 | 10.0 |
| Glycerin (Glycerol EP, vegetable, Spiga Nord) | 5.0 | 5.0 | 5.0 |
| Rhamnolipid | 1.25 | | 1.0 |
| Sophorolipid | | 1.25 | 0.5 |
| Disodium Cocoyl Glutamate (Planatpon ACG LC, BASF) | 2.5 | 2.5 | 2.5 |
| Xanthan Gum (Keltrol CG-SFT, CP Kelco) | 1.2 | 1.2 | 1.2 |
| Stearic Acid (Pristerene 4922, Croda Europe, Ltd.) | 1.2 | 1.2 | 1.2 |
| Citric Acid | ad | ad | ad |
| | pH 5.5 | pH 5.5 | pH 5.5 |
| Olea Europaea Fruit Oil (Cropure Olive, Croda Europe, | 0.2 | 0.2 | 0.2 |
| Ltd.) | | | |
| Glyceryl Oleate (TEGIN ® O V, Evonik) | 1.0 | 1.0 | 1.0 |
| Sodium Cocoyl Glutamate (Plantapon ACG HC, BASF) | 0.8 | 0.8 | 0.8 |
| Desferrioxamine | 0.2 | 0.2 | 0.2 |
| Perfume | 0.1 | 0.1 | 0.1 |
| Preservative | q.s. | q.s. | q.s. |

Recipes 8a, 8b, 8c: Oil Bath

| Ingredients [wt. %] | 8a | 8b | 8c |
|---|---|---|---|
| Water | ad 100.0 | ad 100.0 | ad 100.0 |
| Glycine Soja Oil (Cropure Soybean, Croda Europe, Ltd.) | 20.0 | 20.0 | 20.0 |
| Rhamnolipid | 6.0 | | |
| Sophorolipid | | 7.15 | |
| Glucolipid | | | 7.0 |
| Polyglyceryl-3 Palmitate (Dermofeel ® PP, Evonik Dr. Straetmans) | 4.5 | 4.5 | 4.5 |
| Glyceryl Caprylate (Dermosoft ® GMCY, Evonik Dr. Straetmans) | 3.0 | 3.0 | 3.0 |
| Desferrioxamine | 2.0 | 2.0 | 2.0 |
| Simmondsia Chinensis Seed Oil (AEC Jojoba Oil Refined, A & E | 1.2 | 1.2 | 1.2 |
| Connock, Perfumery & Cosmetics Ltd.) | | | |
| Prunus Amygdalus Dulcis (Sweet Almond) Oil (Cropure Almond, | 1.0 | 1.0 | 1.0 |
| Croda Europe, Ltd.) | | | |
| Triticum Vulgare Germ Oil (Cropure Wheatgerm, Croda Europe, | 0.5 | 0.5 | 0.5 |
| Ltd.) | | | |
| Tocopherol (Euxyl K 700, Schülke & Mayr GmbH) | 0.2 | 0.2 | 0.2 |
| Limonene (Dipentene No. 122, Hercules Inc.) | 0.1 | 0.1 | 0.1 |
| Citral | 0.1 | 0.1 | 0.1 |
| Preservative | q.s. | q.s. | q.s. |
| Dyes | q.s. | q.s. | q.s. |

Recipe 9a, 9b: Oil Bath

| | Ingredients [wt. %] | 9 | 9b |
|---|---|---|---|
| Phase | C12-C15 Alkyl Benzoate (TEGOSOFT ® TN, Evonik) | 29.5 | 29.5 |
| A | Helianthus Annuus (Sunflower) Seed Oil | 26.0 | 26.0 |
| | Paraffinum Perliquidum | 30.0 | 30.0 |
| | Diethylhexyl Sodium Sulfosuccinate (TEGO ® Sulfosuccinate | 9.0 | 9.0 |
| | DO 75; Evonik | | |
| | Polyglyceryl-3 Caprate (TEGOSOFT ® PC 31 MB; Evonik) | 5.0 | 5.0 |
| | Rhamnolipid | 0.45 | — |
| | Sophorolipid | — | 0.6 |
| Phase | Water | Ad 100 | Ad 100 |
| B | | | |
| Phase | Preservative | q.s. | q.s. |
| Z | Desferrioxamine | 0.5 | 0.5 |

Recipes 10a, 10b, 10c: Micellar Water for make-up removal

| Ingredients [wt. %] | 10a | 10b | 10c |
|---|---|---|---|
| Water | ad 100.0 | ad 100.0 | ad 100.0 |
| Perfume | 0.1 | 0.1 | 0.1 |
| Rhamnolipid | 2.5 | | 1.0 |
| Sophorolipid | | 2.0 | 1.5 |
| Polyglyceryl-6 Caprylate; Polyglyceryl-3 Cocoate; | 1.0 | 1.0 | 1.0 |
| Polyglyceryl-4 Caprate; Polyglyceryl-6 Ricinoleate | | | |
| (TEGO ® Solve 61, Evonik) | | | |
| Betaine (TEGO ® Natural Betaine, Evonik) | 2.0 | 2.0 | 2.0 |
| Glycerin (Glycerol EP, vegetable, Spiga Nord) | 1.0 | 1.0 | 1.0 |
| Desferrioxamine | 0.1 | 0.1 | 0.1 |
| Preservative | q.s. | q.s. | q.s. |

Recipes 11a, 11b: Natural Micellar Water

| | Ingredients [wt. %] | 11a | 11b |
|---|---|---|---|
| Phase | Caprylyl/Capryl Glucoside; Aqua; Sodium Cocoyl Glutamate; Glyceryl | 2.5 | 2.5 |
| A | Caprylate; Citric Acid; Polyglyceryl-6 Oleate; Sodium Surfactin | | |
| | (symbio ®solv clear plus MB, Evonik) | | |
| | Perfume | 0.3 | 0.3 |
| | Rhamnolipids | 0.75 | |
| | Sophorolipids | | 0.75 |
| Phase | Glycerin | 5.0 | 5.0 |
| B | Betaine (TEGO ® Natural Betaine; Evonik) | 2.0 | 2.0 |
| | Phenylpropanol (dermosoft ® 250 eco; Evonik) | 0.4 | 0.4 |
| | Pentylene Glycol (dermosoft ® Pentiol eco; Evonik) | 2.0 | 2.0 |
| | Sodium Phytate; Aqua; Alcohol (dermofeel ® PA-3; Evonik) | 0.1 | 0.1 |
| | Desferrioxamine | 0.1 | 0.1 |
| | Water, deionized | Ad 100 | Ad 100 |

Recipes 12a, 12b: Micellar Water

| Ingredients [wt. %] | 12a | 12b |
|---|---|---|
| Rhamnolipids | 2.75 | |
| Sophorolipids | | 3.25 |
| Water | Ad 100 | Ad 100 |
| Betaine (TEGO ® Natural Betaine; Evonik) | 2.0 | 2.0 |
| Pentylene Glycol (dermosoft ® Pentiol eco; Evonik) | 4.0 | 4.0 |
| Sodium Anisate (dermosoft ® anisate eco; Evonik) | 0.3 | 0.3 |
| Glycerin | 1.0 | 1.0 |
| Desferrioxamine | 0.1 | 0.1 |
| Perfume | q.s. | q.s. |

Recipes 13a, 13b, 13c: Skin Tonic

| | Ingredients [wt. %] | 13a | 13b | 13c |
|---|---|---|---|---|
| Phase | Water, deionized | Ad 100 | Ad 100 | Ad 100 |
| A | Glycerin | 4.0 | 4.0 | 4.0 |
| | Sodium PCA; Aqua (Ajidew NL-50N; Ajinomoto) | 0.5 | 0.5 | 0.5 |
| | Desferrioxamine | 0.2 | 0.2 | 0.2 |
| Phase | Rhamnolipids | 0.75 | — | — |
| B | Sophorolipids | — | 0.9 | — |
| | Glucolipids | — | — | 0.75 |
| | Sodium Caproyl/Lauroyl Lactylate; Triethyl Citrate | 1.0 | 1.0 | |
| | (dermosoft ® decalact liquid; Evonik) | | | |
| | Alcohol denat. | 25.0 | 25.0 | 25.0 |
| | Perfume | 0.3 | 0.3 | 0.3 |
| | Triethyl Citrate (dermofeel ® TEC eco; Evonik) | 2.0 | 2.0 | 2.0 |

Recipes 14a, 14b, 14c: Scalp Serum

| | Ingredients [wt. %] | 14a | 14b | 14c |
|---|---|---|---|---|
| Phase | Water | Ad 100 | Ad 100 | Ad 100 |
| A | Propylene Glycol | 1.0 | 1.0 | 1.0 |
| | Xanthan Gum (Keltrol CG-SFT) | 0.2 | 0.2 | 0.2 |
| Phase | Rhamnolipids | 0.25 | — | — |
| B | Sophorolipids | — | 0.3 | — |
| Phase | Glucolipids | — | — | 0.3 |
| C | Ceramide NG; Olea Europaea (Olive) Fruit Oil; Ricinus | 1.0 | 1.0 | 1.0 |
| | Communis (Castor) Seed Oil; Evonik) | | | |
| | Polyglyceryl-3 Caprylate (TEGO ® Cosmo P 813 MB; | 1.0 | 1.0 | 1.0 |
| | Evonik) | | | |
| | Phenoxyethanol; Caprylyl Glycol (Verstatil ® PC; Evonik) | 1.0 | 1.0 | 1.0 |
| | Desferrioxamine | 0.1 | 0.1 | 0.1 |
| Phase | Citric Acid (10% sol.) | q.s. | q.s. | q.s. |
| D | | | | |

Recipes 15a, 15b, 15c: Micellar Cleansing Foam

| | Ingredients [wt. %] | 15a | 15b | 15c |
|---|---|---|---|---|
| Phase | Polyglyceryl-Caprylate; Polyglyceryl-3 Cocoate; Polyglyceryl- | 4.0 | 4.0 | 4.0 |
| A | 4 Caprtar; Polyglyceryl-6 Ricinoleate (TEGO ® Solve 61 MB, | | | |
| | Evonik | | | |
| | Rhamnolipid | 0.5 | | |
| | Sophorolipid | | 0.5 | |
| | Glucolipids | | | 0.5 |
| | Prunus Amygdalus Dulcis (Sweet Almond) Oil | 0.2 | 0.2 | 0.2 |
| | Caprylyl/Capryl Glucoside; Aqua; Sodium Cocoyl Glutamate; | 1.67 | 1.67 | 1.67 |
| | Glyceryl Caprylate; Citric Acid; Polyglyceryl-6 Oleate; | | | |
| | Sodium Surfactin (symbio ®solv clear plus MB, Evonik) | | | |
| Phase | Bisabolol | 0.1 | 0.10 | 0.1 |
| B | Water | Ad 100 | Ad 100 | Ad 100 |
| | Glycerin | 3.0 | 3.0 | 3.0 |
| | Benzyl Alcohol; Caprylyl Glycol; Benzoic Acid (Verstatil ® | 1.0 | 1.0 | 1.0 |
| | BOB, Evonik) | | | |
| | Desferrioxamine | 0.1 | 0.1 | 0.1 |
| | NaOH (10%) | Ad | Ad | Ad |
| | | pH 5.5 | pH 5.5 | pH 5.5 |

Recipes 16a, 16b, 16c: Liquid Facial Cleanser

| Ingredients [wt. %] | 16a | 16b | 16c |
|---|---|---|---|
| Rhamnolipids | 1.5 | — | — |
| Sophorolipids | — | 1.5 | — |
| Glucolipids | | | 1.5 |
| Polyglyceryl-4 Caprate (TEGOSOFT ® | 2.0 | 2.0- | 2.0 |
| PC 41 MB; Evonik) | | | |
| Benzyl Alcohol; Caprylyl Glycol; Benzoic | 2.0 | 2.0 | 2.0 |
| Acid (Verstatil ® BOB, Evonik) | | | |
| Water | Ad 100 | Ad 100 | Ad 100 |
| Glycerin | 2.0 | 2.0 | 2.0 |
| 1,2-Hexanediol (dermosoft ® Hexiol) | 1.4 | 1.4 | 1.4 |
| Sodium Hydroxide (25% sol) | q.s. | q.s. | q.s. |
| Desferrioxamin | 0.2 | 0.2 | 0.2 |

Recipes 17a, 17b, 17c: Biphasic Make-up Remover

| | Ingredients [wt. %] | 17a | 17b | 17c |
|---|---|---|---|---|
| Phase | Water, deionized | Ad 100 | Ad 100 | Ad 100 |
| A | Glycerin (Glycerin 99.5%, Ph. Eur.; Cremer Oleo) | 10.0 | 10.0 | 10.0 |
| | Glycerin; Aqua; Sodium Levulinate; Sodium Anisate | 3.5 | 3.5 | 3.5 |
| | (dermosoft ® 1388 eco) | | | |
| | Polyglyceryl-10 Laurate; Aqua; Citric Acid | 0.45 | 0.45 | 0.45 |
| | (dermofeel ® G 10 L 70 MB; Evonik) | | | |
| | Rhamnolipids | 0.5 | — | — |
| | Sophorolipids | | 0.25 | |
| | Glucolipids | | | 0.3 |
| | Sodium Chloride | 1.0 | 1.0 | 1.0 |
| Phase | Glyceryl Caprylate (dermosoft ® GMCY MB; Evonik) | 0.25 | 0.25 | 0.25 |
| B | Helianthus annuus seed oil (Sunflower oil) (Henry | 42.0 | 42.0 | 42.0 |
| | Lamotte Oils GmbH) | | | |
| | Seabuckthorn Pulp Oil (Hippophae Rhamnoides | 0.1 | 0.1 | 0.1 |
| | Fruit Oil (Henry Lamotte Oils GmbH) | | | |
| | Tocopherol; Helianthus Annuus (Sunflower) Seed | 0.1 | 0.1 | 0.1 |
| | Oil (derrmofeel Toco 70 non GMO; Evonik) | | | |
| | Desferrioxamin | 0.4 | 0.4 | 0.4 |

Recipes 18a, 18b, 18c: Facial Cleansing Gel

| Ingredients [wt. %] | 18a | 18b | 18b |
|---|---|---|---|
| Water | Ad 100 | Ad 100 | Ad 100 |
| Xanthan Gum (Keltrol CG-SFT) | 1.5 | 1.5 | 1.5 |
| Sodium Cocoamphoacetate | 10.0 | 10.0 | 10.0 |
| (REWOTERIC ® AM C MB; Evonik) | | | |
| Citric Acid (30% sol.) | 1.6 | 1.6 | 1.6 |
| Capryl/Capramidopropyl Betaine | 6.6 | 6.6 | 6.6 |
| (TEGO ® Betain 810 MB; Evonik) | | | |
| Rhamolipids | 2.0 | | |
| Sophorolipids | | 1.5 | |
| Glucolipids | | | 1.0 |
| Sodium Chloride | q.s | q.s. | q.s. |
| Sorbitan Caprylate, Glyceryl Oleate | 1.5 | 1.5 | 1.5 |
| (TEGO ® Remo 95 MB) | | | |
| Phenoxyethanol; Benzoic Acid | 0.8 | 0.8 | 0.8 |
| (Verstatil ® BP; Evonik) | | | |
| Desferrioxamine | 0.05 | 0.05 | 0.05 |

Recipes 19a, 19b, 19c: Wet Wipe solution

| Ingredients [wt. %] | 19a | 19b | 19c |
|---|---|---|---|
| Rhamnolipid | 3.5 | — | — |
| Sophorolipid | — | 3.5 | — |
| Glucolipid# | — | — | 2.0 |
| Aloe Barbadensis Leaf Extract (Aloe-Con UP 40, Florida Food | 0.2 | 0.2 | 0.2 |
| Products Inc.) | | | |
| Isopropyl Myristate (TEGOSOFT ® M, Evonik) | 0.2 | 0.2 | 0.2 |
| Disodium Cocoamphodiacetate (REWOTERIC ® AM 2 C NM, | 1.5 | 1.5 | 1.5 |
| Evonik, 39%) | | | |
| Perfume | 0.2 | 0.2 | 0.2 |
| Propylene Glycol (Euxyl K 320, Schülke & Mayr GmbH) | 2.5 | 2.5 | 2.5 |
| Hydrolyzed Silk (Crosilk 10000, Croda Inc.) | 0.2 | 0.2 | 0.2 |
| Caprylyl/Capryl Glucoside (Plantacare 810 UP, BASF) | 0.7 | 0.7 | 0.7 |
| Water | ad 100.0 | ad 100.0 | ad 100.0 |
| Citric Acid | ad | ad | ad |
| | pH 5.0 | pH 5.0 | pH 5.0 |
| Desferrioxamine | 0.5 | 0.5 | 0.5 |
| Phenoxyethanol (S&M Phenoxyethanol, Schülke & Mayr | 0.5 | 0.5 | 0.5 |
| GmbH) | | | |
| Dehydroacetic Acid (Unisept DHA (Universal Preserv-A-Chem, | 0.1 | 0.1 | 0.1 |
| Inc.) | | | |
| Sodium Benzoate (Euxyl K 712, Schülke & Mayr GmbH) | 0.4 | 0.4 | 0.4 |
| Salicylic Acid (Salicylic acid nat.; Evonik Dr. Straetmans | 0.5 | 0.5 | 0.5 |
| GmbH) | | | |

Recipes 20a, 20b, 20c Cleansing Bar

| Ingredients [wt. %] | 20a | 20b | 20b |
|---|---|---|---|
| Sorbitan Oleate (TEGO ® SMO V; Evonik) | 1.00 | 1.00 | 1.00 |
| Theobrom Cacao (Cocoa) Seed Butter | 6.80 | 6.80 | 6.80 |
| Hydrogenated Palm Oil; Hydrogenated Rapeseed | 2.50 | 2.50 | 2.50 |
| Oil (dermofeel ® viscolid MB; Evonik) | | | |
| Cetearyl Alcohol (TEGO ® Alkanol | 24.5 | 24.5 | 24.5 |
| 1618 MB; Evonik) | | | |
| Sorbitan Caprylate; Glyceryl Oleate | 3.00 | 3.00 | 3.00 |
| (TEGO ® Remo 95 MB; Evonik) | | | |
| Cellulose (TEGO ® Feel C 10; Evonik) | 2.50 | 2.50 | 2.50 |
| Sodium Coco-Sulfate, 94% | 25.0 | 25.0 | 25.0 |
| Cocamidopropyl Betaine (TEGO ® Betain | 25.0 | 25.0 | 25.0 |
| CK D MB; Evonik) | | | |
| Rhamnolipid | 0.25 | — | — |
| Soporolipid | — | 0.25 | — |
| Glucolipids | — | — | 0.3 |
| Tocopherol; Helianthus Annuus | 0.2 | 0.2 | 0.2 |
| (Sunflower) Seed Oil | | | |
| (dermofeel ® Toco 70 non GMO; Evonik) | | | |
| Glyceryl Caprylate (dermosoft ® GMCY | 0.5 | 0.5 | 0.5 |
| MB; Evonik) | | | |
| Oleyl Erucate (TEGOSOFT ® OER MB; Evonik) | 6.0 | 6.0 | 6.0 |
| Zinc Oxide | 2.0 | 2.0 | 2.0 |
| Desferrioxamine | 0.4 | 0.4 | 0.4 |
| Perfume | 1.0 | 1.0 | 1.0 |

Recipes 21a: Cleansing Cream

| | Ingredients [wt. %] | 21 |
|---|---|---|
| Phase A | Helianthus Annuus (Sunflower) Seed Oil | 50.0 |
| | Sorbitan Sesquicaprylate (ANTIL ® Soft SC; Evonik) | 7.0 |
| Phase B | Sodium Laureth Sulfate, 28% | 10.7 |
| | Rhamnolipids | 3.5 |
| | Water | Ad 100 |
| | Glycerin | 1.0 |
| | Desferrioxamine | 0.4 |
| Phase C | Preservative, perfume | q.s. |

Recipes 22a, 22b, 22c: Body Wash Peeling

| | Ingredients [wt. %] | 22a | 22b | 22c |
|---|---|---|---|---|
| Phase | Helianthus Annuus (Sunflower) Seed Oil | 50.0 | 50.0 | 50.0 |
| A | Diethylhexyl Sodium Sulfosuccinate (TEGO ® | 9.0 | 9.0 | 9.0 |
| | Sulfosuccinate DO 75; Evonik) | | | |
| Phase | Disodium Laureth Sulfate (REWOPOL ® SB FA 30 | 7.0 | 7.0 | 7.0 |
| B | B MB; Evonik) | | | |
| | Cocamidopropyl Betaine (TEGO ® Betain F 50 MB; | 5.0 | 5.0 | 5.0 |
| | Evonik) | | | |
| | Rhamnolipids | 1.5 | — | |
| | Sophorolipids | — | 1.25 | — |
| | Glucolipids | — | — | 1.5 |
| | Water | Ad 100 | Ad 100 | Ad 100 |
| | Glycerin | 2.0 | 2.0 | 2.0 |
| | Desferrioxamine | 0.4 | 0.4 | 0.4 |
| Phase | Hydrated Silica (Sipernat 2200 PC; Evonik) | q.s. | q.s. | q.s. |
| C | Preservative, perfume | q.s. | q.s. | q.s. |

Recipes 23a, 23b, 23c: Cleansing Powder

| | Ingredients [wt. %] | 23a | 23b | 23b |
|---|---|---|---|---|
| Phase | Capryl/Capramidopropyl Betaine (TEGO ® Betain | 12.0 | 12.0 | 12.0 |
| A | 810 MB; Evonik) | | | |
| | Sodium Cocoamphoacetate (REWOTERIC ® AM C | 15.0 | 15.0 | 15.0 |
| | MB; Evonik) | | | |
| | Sucrose Cocoate (TEGOSOFT ® LSE 65 K Soft) | 3.0 | 3.0 | 3.0 |
| | Sorbitan Sesquicaprylate (ANTIL ® Soft SC MB; | 1.0 | 1.0 | 1.0 |
| | Evonik) | | | |
| | Bis-(Isostearoyl/Oleoyl Isopropyl) imonium | 1.0 | 1.0 | 1.0 |
| | Methosulfate (VARISOFT ® EQ 100; Evonik) | | | |
| | Rhamnolipids | 2.5 | 1.0 | |
| | Sophorolipids | | 1.0 | |
| | Glucolipids | | | 2.0 |
| | Oleyl Erucate (TEGOSOFT ® OER MB; Evonik) | 2.0 | 2.0 | 2.0 |
| | Helianthus Annuus (Sunflower) Seed Oil | 5.0 | 5.0 | 5.0 |
| | Citric Acid (30% sol.) | 3.0 | 3.0 | 3.0 |
| Phase | Sipernat 500 LS Hydrated Silica) | | | |
| B | Wheat Flour | 29.0 | 29.0 | 29.0 |
| | Cocamidopropyl Betaine (TEGO ® Betain CK D MB; | 12.5 | 12.5 | 12.5 |
| | Evonik) | | | |
| | Desferrioxamine | 0.2 | 0.2 | 0.2 |
| Phase | Preservative, Perfume | q.s. | q.s. | q.s. |
| C | | | | |

Recipes 24a, 24b, 24c: Cleansing Oil

| Ingredients [wt. %] | 24a | 24b | 24c |
|---|---|---|---|
| Rhamnolipids | 0.25 | — | — |
| Sophorolipids | — | 0.15 | — |
| Glucolipids | — | — | 0.1 |
| Desferrioxamine | 0.1 | 0.1 | 0.1 |
| Polyglyceryl-2 Sesquioleate (dermofeel ® GO soft; | 5.0 | 5.0 | 5.0 |
| Evonik) | | | |
| Helianthus Annuus (Sunflower) Seed Oil | 36.0 | 36.0 | 36.0 |
| Isoamyl Laurate (dermofeel ® sensolv MB; Evonik) | 35.0 | 35.0 | 35.0 |
| TEGOSOFT ® DC MB (Decyl Cocoate; Evonik) | 5 | 5 | 5 |
| Polyglyceryl-2 Caprate (dermosoft ® DGMC MB; | 2 | 2 | 2 |
| Evonik) | | | |
| Tocopherol; Helianthus Annuus | 1.0 | 1.0 | 1.0 |
| (Sunflower) Seed Oil | | | |
| (dermofeel ® Toco 70 non GMO; Evonik) | | | |
| Sesamum Indicum (Sesame Seed) Oil | 16 | 16 | 16 |

Recipes 25a, 25b, 25c: Anti-Transpirant Deo

| | Ingredients [wt. %] | 25a | 25b | 25c |
|---|---|---|---|---|
| Phase | Rhamnolipid | 0.5 | | |
| A | Sophorolipid | | 0.5 | |
| | Glucolipid | | | 0.5 |
| | Dicaprylyl Ether (Cetiol OE, BASF) | 0.3 | 0.3 | 0.3 |
| | Geraniol (Nerol 800, International Flavors & | 0.1 | 0.1 | 0.1 |
| | Fragrances Inc.) | | | |
| | Linalool (Lipofresh, Lipo Chemicals, Inc.) | 0.1 | 0.1 | 0.1 |
| | Perfume | 0.1 | 0.1 | 0.1 |
| Phase | Propylene Glycol (Euxyl K 320, Schülke & Mayr | 1.0 | 1.0 | 1.0 |
| B | GmbH) | | | |
| | Butylene Glycol (Oxea Corparation) | 0.2 | 0.2 | 0.2 |
| | Water | 5.0 | 5.0 | 5.0 |
| | Palmitamidopropyltrimonium Chloride (VARISOFT ® | 1.0 | 1.0 | 1.0 |
| | PATC, Evonik) | | | |
| | Water | 50.0 | 50.0 | 50.0 |
| Phase | Hydroxethyl Ethylcellulose (Structure Cel 4400 E, | 0.8 | 0.8 | 0.8 |
| C | AkzoNobel) | | | |
| | Sodium Hydroxide (10% in water) | 0.3 | 0.3 | 0.3 |
| Phase | Aluminium Chlorohydrate (Locron L, Clariant) | 15.0 | 15.0 | 15.0 |
| D | | | | |
| Phase | Preservative | q.s. | q.s. | q.s. |
| E | Desferrioxamine | 0.1 | 0.1 | 0.1 |
| | Water | ad 100.0 | ad 100.0 | ad 100.0 |

Recipes 26a, 26b, 26c: Mouthwash

| Ingredients [wt. %] | 26a | 26b | 26c |
|---|---|---|---|
| Rhamnolipid | 0.5 | | 0.25 |
| Sophorolipid | | 0.6 | 0.3 |
| Essential Oil | 0.1 | 0.1 | 0.1. |
| Flavor | 0.2 | 0.2 | 0.2 |
| Water | ad 100.0 | ad 100.0 | ad 100.0 |
| Sorbitol (Karion FP Liquid, Merck) | 3.0 | 3.0 | 3.0 |
| Desferrioxamine | 0.1 | 0.1 | 0.1 |
| Preservative | q.s. | q.s. | q.s. |
| Dyes | q.s. | q.s. | q.s. |

Recipes 27a, 27b, 27c: Toothpaste

| | Ingredients [wt. %] | 27a | 27b | 27c |
|---|---|---|---|---|
| Phase | Sorbitol (Karion FP Liquid, Merck) | 50.0 | 50.0 | 50.0 |
| A | Water | ad 100.0 | ad 100.0 | ad 100.0 |
| | Rhamnolipid | 0.75 | | |
| | Sophorolipid | | 0.75 | |
| | Glucolipid | | | 1.5 |
| | Sodium Carboxymethylcellulose (Blanose 7MXF, | 1.2 | 1.2 | 1.2 |
| | Ashland) | | | |
| Phase | Sodium Saccharine (Sigma Aldrich) | 0.1 | 0.1 | 0.1 |
| B | Sodium Fluoride (Sigma Aldrich) | 0.1 | 0.1 | 0.1 |
| Phase | Titanium Dioxide (Caesar & Loretz) | 0.4 | 0.4 | 0.4 |
| C | Hydrated Silica (Zeodent ® 113, Evonik) | 14.0 | 14.0 | 14.0 |
| | Hydrated Silica (Zeodent ® 165, Evonik) | 8.0 | 8.0 | 8.0 |
| Phase | Flavor oil | 1.0 | 1.0 | 1.0 |
| D | | | | |
| Phase | Desferrioxamine | 0.2 | 0.2 | 0.2 |
| E | | | | |
| Phase | Glyceryl Caprylate (dermosoft ® GMCY, Evonik) | 0.3 | 0.3 | 0.3 |
| F | | | | |

Recipes 28a, 28b, 28c: Kitchen Cleaning Spray

| Ingredient | 28a | 28b | 28c |
|---|---|---|---|
| REWOTERIC ® AM V | 0.5 | 0.5 | 0.5 |
| Rhamnolipid | 0.5 | | |
| Sophorolipid | | 0.5 | |
| Glucolipid | | | 1.0 |
| TOMAKLEEN ® G-14 | 2.0 | 2.0 | 2.0 |
| Water (Dye. Perfume) | 89.5 | 89.5 | 89.5 |
| Desferrioxamine | 3.0 | 3.0 | 3.0 |
| Triethanolamine | 4.0 | 4.0 | 4.0 |

Recipes 29a, 29b, 29c: Extra Mild Dish Wash Foam

| Ingredient | 29a | 29b | 29c |
|---|---|---|---|
| C10-16 Alkylpolyglucoside. 50 | 10.0 | 10.0 | 10.0 |
| Xanthan Gum | 0.1 | 0.1 | 0.1 |
| Glycerin | 2.0 | 2.0 | 2.0 |
| Water | 76.9 | 76.9 | 76.9 |
| Rhamnolipid | 0.5 | | |
| Sophorolipid | | 0.5 | |
| Glucolipid | | | 1.0 |
| Sophorolipid | 2.0 | 2.0 | 2.0 |
| REWOPOL ® SB CS 50 | 5.0 | 5.0 | 5.0 |
| TEGO ® Betain C 60 | 3.0 | 3.0 | 3.0 |
| Desferrioxamine | 0.5 | 0.5 | 0.5 |
| Preservative | qs. | qs. | qs. |

Recipes 30a, 30b, 30c: Automatic Rinse Aid for Direct Use 1

| Ingredient | 30a | 30b | 30c |
|---|---|---|---|
| TEGOTENS ® EC 11 | 5.0 | 5.0 | 5.0 |
| Rhamnolipid | 2.5 | | |
| Sophorolipid | | 2.5 | |
| Glucolipid | | | 5.0 |
| Citric acid monohydrate | 5.0 | 5.0 | 5.0 |
| Desferrioxamine | 0.2 | 0.2 | 0.2 |
| Water (Dye. Perfume) | 85.0 | 85.0 | 85.0 |

Recipes 31a, 31b, 31c: Automatic Rinse Aid for Direct Use 2

| Ingredient | 31a | 31b | 31c |
|---|---|---|---|
| Citric acid monohydrate | 5.0 | 5.0 | 5.0 |
| Sodium Cumene Sulfonate. 92 | 1.5 | 1.5 | 1.5 |
| Water (Dye; Perfume) | 83.5 | 83.5 | 83.5 |
| TEGOTENS ® EC 11 | 6.0 | 6.0 | 6.0 |
| Rhamnolipid | 2.0 | | |
| Sophorolipid | | 2.0 | |
| Glucolipid | | | 4.0 |
| Desferrioxamine | 0.2 | 0.2 | 0.2 |

Recipes 32a, 32b, 32c: Automatic Rinse Aid for Direct Use 3

| Ingredient | 32a | 32b | 32c |
|---|---|---|---|
| Citric acid monohydrate | 5.0 | 5.0 | 5.0 |
| Sodium Cumene Sulfonate. 92 | 1.5 | 1.5 | 1.5 |
| Water (Dye. Perfume) | 83.5 | 83.5 | 83.5 |
| TEGOTENS ® EC 11 | 7.0 | 7.0 | 7.0 |
| Rhamnolipid | 1.5 | | |
| Sophorolipid | | 1.5 | |
| Glucolipid | | | 3.0 |
| Desferrioxamine | 0.5 | 0.5 | 0.5 |

Recipes 33a, 33b, 33c: Glass Cleaner
With Optimized Antifogging Efficiency

| Ingredient | 33a | 33b | 33c |
|---|---|---|---|
| REWOPOL ® TS 35 | 1.0 | 1.0 | 1.0 |
| Rhamnolipid | 0.1 | | |
| Sophorolipid | | 0.1 | |
| Glucolipid | | | 0.2 |
| Isopropanol | 15.0 | 15.0 | 15.0 |
| EDTA | 1.0 | 1.0 | 1.0 |
| Desferrioxamin | 0.5 | 0.5 | 0.5 |
| Water (Dye. Perfume) | 81.8 | 81.8 | 81.8 |

Recipes 34a, 34b, 34c: Oven Cleanser for Smoking Chamber

| Ingredient | 34a | 34b | 34c |
|---|---|---|---|
| Water | 77.6 | 77.6 | 77.6 |
| Trilon A liquid | 8.0 | 8.0 | 8.0 |
| Sodium carbonate | 6.0 | 6.0 | 6.0 |
| REWOTERIC ® AM KSF 40 | 5.0 | 5.0 | 5.0 |
| Laureth-6 | 1.2 | 1.2 | 1.2 |
| Rhamnolipid | 0.6 | | |
| Sophorolipid | | 0.6 | |
| Glucolipid | | | 1.2 |
| Desferrioxamine | 0.08 | 0.08 | 0.08 |
| DOWANOL DPnB | 1.0 | 1.0 | 1.0 |

Recipes 35a, 35b, 35c: All Purpose Cleanser (Microemulsion)

| Ingredient | 35a | 35b | 35c |
|---|---|---|---|
| Water | 73.0 | 73.0 | 73.0 |
| TEGOTENS ® AM VSF | 6.0 | 6.0 | 6.0 |
| Rhamnolipid | 1.0 | | |
| Sophorolipid | | 1.0 | |
| Glucolipid | | | 2.0 |
| REWOPOL ® D 510 NC | 5.0 | 5.0 | 5.0 |
| Undeceth-6 | 3.0 | 3.0 | 3.0 |
| Potassium hydroxide. 50 | 5.0 | 5.0 | 5.0 |
| Chelating Agent. MGDA | 5.0 | 5.0 | 5.0 |
| Desferrioxamine | 1.0 | 1.0 | 1.0 |

Recipes 36a, 36b, 36c: Low Foaming Hard Surface Degreaser

| Ingredient | 36a | 36b | 36c |
|---|---|---|---|
| C9-11 FATTY ALCOHOL ETHOXYLATE - 8 | 2.0 | 2.0 | 2.0 |
| MOLES OF EO | | | |
| Rhamnolipid | 0.5 | | |
| Sophorolipid | | 0.5 | |
| Glucolipid | | | 1.0 |
| TEGOTENS ® G 826 C | 4.0 | 4.0 | 4.0 |
| Chelating Agent. MGDA | 7.5 | 7.5 | 7.5 |
| Water (Dye. Perfume) | 85.5 | 85.5 | 85.5 |

Recipes 37a, 37b, 37c: Low Foaming
Hard Surface Degreaser (101630-23)

| Ingredient | 37a | 37b | 37c |
|---|---|---|---|
| C9-11 FATTY ALCOHOL ETHOXYLATE - 8 | 2.0 | 2.0 | 2.0 |
| MOLES OF EO | | | |
| Rhamnolipid | 0.5 | | |
| Sophorolipid | | 0.5 | |
| Glucolipid | | | 1.0 |
| TEGOTENS ® G 826 C | 4.0 | 4.0 | 4.0 |
| Desferrioxamine | 1 | 1 | 1 |
| Water (Dye. Perfume) | 85.5 | 85.5 | 85.5 |

Recipes 338a, 38b, 38c: Foaming Hard Surface Degreaser 1

| Ingredient | 38a | 38b | 38c |
|---|---|---|---|
| C9-11 FATTY ALCOHOL ETHOXYLATE - 8 | 2.0 | 2.0 | 2.0 |
| MOLES OF EO | | | |
| TEGOTENS ® AM VSF | 6.0 | 6.0 | 6.0 |
| Rhamnolipid | 0.5 | | |
| Sophorolipid | | 0.5 | |
| Glucolipid | | | 1.0 |
| Desferrioxamine | 1.5 | 1.5 | 1.5 |
| Water (Dye. Perfume) | 83.5 | 83.5 | 83.5 |

Recipes 39a, 39b, 39c: Foaming Hard Surface Degreaser 2

| Ingredient | 39a | 39b | 39c |
|---|---|---|---|
| C9-11 FATTY ALCOHOL ETHOXYLATE - 8 | 3.0 | 3.0 | 3.0 |
| MOLES OF EO | | | |
| REWOTERIC ® AM KSF 40 | 2.5 | 2.5 | 2.5 |
| Rhamnolipid | 0.5 | | |
| Sophorolipid | | 0.5 | |
| Glucolipid | | | 1.0 |
| Desferrioxamine | 1.5 | 1.5 | 1.5 |
| Water (Dye. Perfume) | 83.5 | 83.5 | 83.5 |

Recipes 40a, 40b, 40c: Low Foaming Hard Surface Degreaser 3

| Ingredient | 40a | 40b | 40c |
|---|---|---|---|
| Rhamnolipid | 0.5 | | |
| Sophorolipid | | 0.5 | |
| Glucolipid | | | 1.0 |
| C8-10 Alkylpolyglucosid | 4.0 | 4.0 | 4.0 |
| Sodium C13-17 Alkane Sulfonate. 30 | 2.0 | 2.0 | 2.0 |
| Desferrioxamine | 1.5 | 1.5 | 1.5 |
| Water (Dye. Perfume) | 85.5 | 85.5 | 85.5 |

Recipes 41a, 41b, 41c: Low Foaming Hard Surface Degreaser 4

| Ingredient | 41a | 41b | 41c |
|---|---|---|---|
| Rhamnolipid | 0.5 | | |
| Sophorolipid | | 0.5 | |
| Glucolipid | | | 1.0 |
| C8-10 Alkylpolyglucosid | 4.0 | 4.0 | 4.0 |
| Sodium C13-17 Alkane Sulfonate. 30 | 2.0 | 2.0 | 2.0 |
| Chelating Agent. MGDA | 7.0 | 7.0 | 7.0 |
| Water (Dye. Perfume) | 85.5 | 85.5 | 85.5 |

Recipes 42a, 42b, 42c: Low Foaming Hard Surface Degreaser 4

| Ingredient | 42a | 42b | 42c |
|---|---|---|---|
| Rhamnolipid | 0.5 | | |
| Sophorolipid | | 0.5 | |
| Glucolipid | | | 1.0 |
| C8-10 Alkylpolyglucosid | 4.0 | 4.0 | 4.0 |
| Sodium C13-17 Alkane Sulfonate. 30 | 2.0 | 2.0 | 2.0 |
| Desferrioxamine | 10.0 | 10.0 | 10.0 |
| Water (Dye. Perfume) | 85.5 | 85.5 | 85.5 |

Recipes 43a, 43b, 43c: Hard Surface
Degreaser From Renewable Surfactants

| Ingredient | 43a | 43b | 43c |
|---|---|---|---|
| Water | 89.3 | 89.3 | 89.3 |
| Sodium carbonate | 3.0 | 3.0 | 3.0 |
| TEGOTENS ® AM VSF | 2.4 | 2.4 | 2.4 |
| Desferrioxamine | 2.0 | 2.0 | 2.0 |
| Laureth-6 | 0.6 | 0.6 | 0.6 |
| Rhamnolipid | 0.2 | | |
| Sophorolipid | | 0.2 | |
| Glucolipid | | | 0.4 |
| TEGOR Polish Additiv Q 70 | 0.3 | 0.3 | 0.3 |

Recipes 44a, 44b, 44c: Highly Efficient Floor Cleanser

| Ingredient | 44a | 44b | 44c |
|---|---|---|---|
| Water | 89.3 | 89.3 | 89.3 |
| Desferrioxamine | 4.0 | 4.0 | 4.0 |
| Sodium carbonate | 3.0 | 3.0 | 3.0 |
| TEGOTENS ® AM VSF | 2.4 | 2.4 | 2.4 |
| Laureth-6 | 0.6 | 0.6 | 0.6 |
| Rhamnolipid | 0.2 | | |
| Sophorolipid | | 0.2 | |
| Glucolipid | | | 0.4 |
| TEGO ® Polish Additiv Q 70 | 0.3 | 0.3 | 0.3 |

Recipes 45a, 45b, 45c: Super Natural
Wash Lotion for Textile Face Masks

| Ingredient | 45a | 45b | 45c |
|---|---|---|---|
| Xanthan Gum | 0.1 | 0.1 | 0.1 |
| Glycerin | 2.0 | 2.0 | 2.0 |
| Water | 76.9 | 76.9 | 76.9 |
| Rhamnolipid | 0.5 | | |
| Sophorolipid | | 0.5 | |
| Glucolipid | | | 1.0 |
| Sophorolipid | 2.0 | 2.0 | 2.0 |
| REWOPOL ® SB CS 50 | 5.0 | 5.0 | 5.0 |
| C8-10 Alkylpolyglucosid | 10.0 | 10.0 | 10.0 |
| TEGO ® Betain C 60 | 3.0 | 3.0 | 3.0 |
| Desferrioxamine | 1.0 | 1.0 | 1.0 |
| Preservative | qs. | qs. | qs. |

Recipes 46, 46b, 46c: Highly Effective Presoaker

| Ingredient | 46a | 46b | 46c |
|---|---|---|---|
| Water | 77.5 | 77.5 | 77.5 |
| Sodium metasilicate | 2.0 | 2.0 | 2.0 |
| Sodium gluconate | 2.0 | 2.0 | 2.0 |
| Sodium carbonate | 2.0 | 2.0 | 2.0 |
| Desferrioxamine | 2.0 | 2.0 | 2.0 |
| TEGOTENS ® AM VSF | 6.5 | 6.5 | 6.5 |
| Undeceth-6 | 0.6 | 0.6 | 0.6 |
| Rhamnolipid | 0.2 | | |
| Sophorolipid | | 0.2 | |
| Glucolipid | | | 0.4 |
| DOWANOL DPnB | 1.0 | 1.0 | 1.0 |

Recipes 47, 47b, 47c: Presoaker (Basic Formula)

| Ingredient | 47a | 47b | 47c |
|---|---|---|---|
| Water | 82.6 | 82.6 | 82.6 |
| Trilon A 92 | 3.0 | 3.0 | 3.0 |
| Desferrioxamine | 2.0 | 2.0 | 2.0 |
| Sodium metasilicate | 2.0 | 2.0 | 2.0 |
| Sodium carbonate | 2.0 | 2.0 | 2.0 |
| Bayhibit AM | 1.0 | 1.0 | 1.0 |
| REWOTERIC ® AM KSF 40 | 4.0 | 4.0 | 4.0 |
| C9-11 FATTY ALCOHOL ETHOXYLATE - 8 | 1.2 | 1.2 | 1.2 |
| MOLES OF EO | | | |
| Rhamnolipid | 0.6 | | |
| Sophorolipid | | 0.6 | |
| Glucolipid | | | 1.2 |
| DOWANOL DPnB | 3.0 | 3.0 | 3.0 |

Recipes 48a, 48b, 48c: Good Dispersing Presoaker

| Ingredient | 48a | 48b | 48c |
|---|---|---|---|
| Water | 80.0 | 80.0 | 80.0 |
| Trilon A 92 | 3.0 | 3.0 | 3.0 |
| Sodium metasilicate | 2.0 | 2.0 | 2.0 |
| Sodium carbonate | 2.0 | 2.0 | 2.0 |
| Sequion 10 Na 430 | 2.0 | 2.0 | 2.0 |
| TEGOTENS ® 475 | 5.0 | 5.0 | 5.0 |
| C9-11 FATTY ALCOHOL ETHOXYLATE - 7 | 1.5 | 1.5 | 1.5 |
| MOLES OF EO | | | |
| Rhamnolipid | 0.75 | | |
| Sophorolipid | | 0.75 | |
| Glucolipid | | | 1.5 |
| Desferrioxamine | 0.5 | 0.5 | 0.5 |
| DOWANOL PnB | 3.0 | 3.0 | 3.0 |

Recipes 49a, 49b, 49c: Cost Efficient Presoaker

| Ingredient | 49a | 49b | 49c |
|---|---|---|---|
| Water | 80.5 | 80.5 | 80.5 |
| Chelating Agent. M | 8.0 | 8.0 | 8.0 |
| Sodium carbonate | 6.0 | 6.0 | 6.0 |
| TEGOTENS ® AM VSF | 3.5 | 3.5 | 3.5 |
| Laureth-6 | 0.6 | 0.6 | 0.6 |
| Rhamnolipid | 0.2 | | |
| Sophorolipid | | 0.2 | |
| Glucolipid | | | 0.4 |
| Desferrioxamine | 0.2 | 0.2 | 0.2 |
| DOWANOL DPnB | 1.0 | 1.0 | 1.0 |

Recipes 50a, 50b, 50c: Rinse Aid to minimize remaining water

| Ingredient | 50a | 50b | 50c |
|---|---|---|---|
| REWOPAL ® MPG 40 | 6.0 | 6.0 | 6.0 |
| Butyl Cellusolve (BG) | 14.0 | 14.0 | 14.0 |
| Rhamnolipid | 1.5 | | |
| Sophorolipid | | 1.5 | |
| Glucolipid | | | 3.0 |
| Desferrioxamine | 1.0 | 1.0 | 1.0 |
| REWOQUAT ® CR 3099 | 10.0 | 10.0 | 10.0 |
| TEGOSOFT ® OP | 5.0 | 5.0 | 5.0 |
| Water | 62.0 | 62.0 | 62.0 |

Recipes 51a, 51b, 51c: Rinse Aid with Optimal Oil Content

| Ingredient | 51a | 51b | 51c |
|---|---|---|---|
| DOWANOL DPnB | 8.6 | 8.6 | 8.6 |
| REWOPAL ® MPG 40 | 6.2 | 6.2 | 6.2 |
| REWOQUAT ® CR 3099 | 10.0 | 10.0 | 10.0 |
| REWOCARE ® DOC | 4.0 | 4.0 | 4.0 |
| TEGOSOFT ® OP | 3.0 | 3.0 | 3.0 |
| Rhamnolipid | 0.5 | | |
| Sophorolipid | | 0.5 | |
| Glucolipid | | | 1.0 |
| Desferrioxamin | 0.5 | 0.5 | 0.5 |
| Water | 66.8 | 66.8 | 66.8 |
| TEGOPREN ® 6923 | 0.4 | 0.4 | 0.4 |

Recipes 52a, 52b, 52c: Low Foaming All Purpose Cleaner

| Ingredient | 52a | 52b | 52c |
|---|---|---|---|
| Water | 82.0 | 82.0 | 82.0 |
| Trilon A 92 | 3.0 | 3.0 | 3.0 |
| Sodium metasilicate | 2.0 | 2.0 | 2.0 |
| Sodium carbonate | 2.0 | 2.0 | 2.0 |
| Sequion 10 Na 430 | 2.0 | 2.0 | 2.0 |
| Desferrioxamine | 2.0 | 2.0 | 2.0 |
| TEGOTENS ® 475 | 5.0 | 5.0 | 5.0 |
| C9-11 FATTY ALCOHOL ETHOXYLATE - 7 | 1.5 | 1.5 | 1.5 |
| MOLES OF EO | | | |
| Rhamnolipid | 0.75 | | |
| Sophorolipid | | 0.75 | |
| Glucolipid | | | 1.5 |
| DOWANOL PnB | 1.0 | 1.0 | 1.0 |

Recipes 53a, 53b, 3c: Low Foaming Alkaline Cleanser

| Ingredient | 53a | 53b | 53c |
|---|---|---|---|
| Water | 85.5 | 85.5 | 85.5 |
| Chelating Agent. M | 4.0 | 4.0 | 4.0 |
| Sodium carbonate | 6.0 | 6.0 | 6.0 |
| TEGOTENS ® AM VSF | 3.5 | 3.5 | 3.5 |
| Laureth-6 | 0.6 | 0.6 | 0.6 |
| Rhamnolipid | 0.2 | | |
| Sophorolipid | | 0.2 | |
| Glucolipid | | | 0.4 |
| Desferrioxamine | 0.05 | 0.05 | 0.05 |

Recipes 54a, 54b, 4c: Low Foaming Alkaline Cleaner

| Ingredient | 54a | 54b | 54c |
|---|---|---|---|
| Water | 85.35 | 85.35 | 85.35 |
| TOMAKLEEN G-14 | 0.75 | 0.75 | 0.75 |
| TEGOTENS ® AM VSF | 3.5 | 3.5 | 3.5 |
| Desferrioxamine | 2.0 | 2.0 | 2.0 |
| Rhamnolipid | 0.2 | | |
| Sophorolipid | | 0.2 | |
| Glucolipid | | | 0.4 |
| Sodium carbonate | 6.0 | 6.0 | 6.0 |

Recipes 55a, 55b, 55c: Alkaline Cleanser (Automatic Wash)

| Ingredient | 55a | 55b | 55c |
|---|---|---|---|
| Water | 59.0 | 59.0 | 59.0 |
| Tetra Potassium Pyrophosphate (TKPP) | 15.0 | 15.0 | 15.0 |
| Trilon A liquid | 5.0 | 5.0 | 5.0 |
| KOH. 45 | 4.0 | 4.0 | 4.0 |
| Sequion 10 Na 430 | 10.0 | 10.0 | 10.0 |
| Sodium metasilicate | 6.0 | 6.0 | 6.0 |
| Rhamnolipid | 0.5 | | |
| Sophorolipid | | 0.5 | |
| Glucolipid | | | 1.0 |
| Desferrioxamine | 0.2 | 0.2 | 0.2 |

Recipes 56a, 56b, 6c: Metal Cleaner

| Ingredient | 56a | 56b | 56c |
|---|---|---|---|
| Water | 82.0 | 82.0 | 82.0 |
| TOMAKLEEN ® G-14 | 12.0 | 12.0 | 12.0 |
| REWOTERIC ® AM V | 1.5 | 1.5 | 1.5 |
| Rhamnolipid | 1.5 | | |
| Sophorolipid | | 1.5 | |
| Glucolipid | | | 3.0 |
| Desferrioxamine | 1.5 | 1.5 | 1.5 |
| Triethanolamine | 1.5 | 1.5 | 1.5 |

Recipes 57a, 57b, 57c: Metal Cleanser

| Ingredient | 67a | 57b | 57c |
|---|---|---|---|
| Water (Dye. Perfume) | 82.0 | 82.0 | 82.0 |
| Triethanolamine | 1.5 | 1.5 | 1.5 |
| DOWANOL PnP | 6.0 | 6.0 | 6.0 |
| REWOTERIC ® AM V | 1.5 | 1.5 | 1.5 |
| Rhamnolipid | 1.5 | | |
| Sophorolipid | | 1.5 | |
| Glucolipid | | | 3.0 |
| Desferrioxaminef | 1.0 | 1.0 | 1.0 |
| C8-10 Fatty alcohol · 5 EO | 6.0 | 6.0 | 6.0 |

Recipes 58a, 58b, 58c: Cost Efficient Facade Cleanser

| Ingredient | 58a | 58b | 58c |
|---|---|---|---|
| Water | 86.6 | 86.6 | 86.6 |
| Chelating Agent. MGDA | 4.0 | 4.0 | 4.0 |
| Sodium carbonate | 6.0 | 6.0 | 6.0 |
| TEGOTENS ® AM VSF | 2.4 | 2.4 | 2.4 |
| Laureth-6 | 0.6 | 0.6 | 0.6 |
| Rhamnolipid | 0.2 | | |
| Sophorolipid | | 0.2 | |
| Glucolipid | | | 0.4 |
| Desferrioxamine | 0.05 | 0.05 | 0.05 |

Recipes 59a, 59b, 59c: Anti-dust Glass and Window Cleaner 1

| Ingredient | 59a | 59b | 59c |
|---|---|---|---|
| Water | ad. 100 | ad. 100 | ad. 100 |
| Ethanol | 15 | 15 | 15 |
| Rewocare ® 755 | 0.5 | 0.5 | 0.5 |
| Rhamnolipid | 1.0 | | |
| Sophorolipid | | 1.0 | |
| Glucolipid | | | 2.0 |
| Desferrioxamine | 0.01 | 0.01 | 0.01 |
| 2-Phenoxyethanol | 0.1 | 0.1 | 0.1 |
| pH value (adjust to) | 8 | 8 | 8 |

Recipes 60a, 60b, 60c: Anti-dust Glass and Window Cleaner 2

| Ingredient | 60a | 60b | 60c |
|---|---|---|---|
| Water | ad. 100 | ad. 100 | ad. 100 |
| Ethanol | 15 | 15 | 15 |
| Rewocare ® 755 | 0.5 | 0.5 | 0.5 |
| Rhamnolipid | 0.5 | | |
| Sophorolipid | | 0.5 | |
| Glucolipid | | | 1.0 |
| Tegotens ® AM VSF | 1 | 1 | 1 |
| Desferrioxamine | 0.01 | 0.01 | 0.01 |
| 2-Phenoxyethanol | 0.1 | 0.1 | 0.1 |
| pH value (adjust to) | 8.00 | 8.00 | 8.00 |

Recipes 61a, 61b, 61c: Anti-dust Glass and Window Cleaner 3

| Ingredient | 61a | 61b | 61c |
|---|---|---|---|
| Water | ad. 100 | ad. 100 | ad. 100 |
| Ethanol | 3 | 3 | 3 |
| Rhamnolipid | 0.75 | | |
| Sophorolipid | | 0.75 | |
| Glucolipid | | | 1.5 |
| Tegotens ® DO 40 | 1 | 1 | 1 |
| Tegopren ® 5840 | 0.05 | 0.05 | 0.05 |
| Desferrioxamine | 0.05 | 0.05 | |
| 2-Phenoxyethanol | 0.1 | 0.1 | 0.1 |
| pH value (adjust to) | 6.0-7.0 | 6.0-7.0 | 6.0-7.0 |

Recipes 62, 63, 64: All-purpose Cleaner concentrate

| Ingredient | 62 | 63 | 64 |
|---|---|---|---|
| Water | ad. 100 | ad. 100 | ad. 100 |
| Ethanol | 3 | 3 | 5 |
| Rhamnolipid | | | 1 |
| Tego ® Betain Powder MB | | 1 | |
| Rewocare ® 755 | 0.5 | 0.5 | |
| Rewopol ® SB DO 75 | 3 | | 1.5 |
| Sophorolipid | 0.5 | 0.5 | |
| Desferrioxamine | 0.2 | 0.2 | 0.2 |
| Tegotens ® AM VSF | | 3 | |
| 2-Phenoxyethanol | 0.1 | 0.1 | 0.1 |
| pH value (adjust to) | 8 | 8 | 8 |

Recipes 65, 66, 67: All-purpose Cleaner Cleaner Trigger spray

| Ingredient | 65 | 66 | 67 |
|---|---|---|---|
| Water | ad. 100 | ad. 100 | ad. 100 |
| Ethanol | 3 | 3 | 3 |
| Rhamnolipid | | 1.5 | 0.75 |
| Tego ® Betain Powder MB | | | 1 |
| Rewocare ® 755 | | | 0.5 |
| Sophorolipid | 1 | | |
| Desferrioxamine | 0.2 | 0.2 | 0.2 |
| Tegotens ® AM VSF | 1 | | |
| 2-Phenoxyethanol | 0.1 | 0.1 | 0.1 |
| pH value (adjust to) | 8.00 | 8.00 | 8.00 |

Recipes 69, 70: Floor Cleaner

| Ingredient | 68 | 69 | 70 |
|---|---|---|---|
| Water | 93 | 93 | 80 |
| Sophorolipid | | | 0.5 |
| Rhamnolipid | 0.5 | 0.5 | |
| Desferrrioxamine | 0.5 | 0.5 | 0.5 |
| Tego ® PP 1027 | 0.5 | 0.5 | |
| Tego ® Betain Powder MB | 1 | 1 | 1 |
| Tegotens ® AM VSF | | | 3 |
| Tomadol 91-6 | 4 | 4 | 4 |
| Rewocare ® 755 | | | 0.5 |
| 2-Phenoxyethanol | 0.1 | 0.1 | 0.1 |
| pH value (adjust to) | 8.00 | 9.00 | 8.00 |

Recipes 71, 72, 73: Bathroom Cleaner

| Ingredient | 71 | 72 | 73 |
|---|---|---|---|
| Water | ad. 100 | ad. 100 | ad. 100 |
| Rhamnolipid | | 0.75 | |
| Tego ® Betain Powder MB | 1.5 | 1.5 | 1.5 |
| Rewocare ® 755 | 0.5 | 0.5 | |
| Rewopol ® SB DO 75 | | | 1.5 |
| Sophorolipid | 0.5 | 0.25 | 0.25 |
| Tegotens ® AM VSF | 3 | 1.5 | |
| Rewoteric ® AM V | | | 1.5 |
| Tego ® PP 1027 | | | 0.5 |
| Desferrioxamine | 0.25 | 0.25 | 0.25 |
| 2-Phenoxyethanol | 0.1 | 0.1 | 0.1 |
| pH value (adjust to) | 5.5 | 5.5 | 5.5 |

Recipes 74, 75, 76: Handdishwash Liquid

| Ingredient | 75 | 75 | 76 |
|---|---|---|---|
| Water | ad. 100 | ad. 100 | ad. 100 |
| Texapon 70 | 15 | 5 | |
| TEGO ® Betain C 60 | 4.8 | 3 | 3 |
| Rhamnolipid | 1.3 | 1 | 1 |
| Sophorolipid | | 1.3 | |
| Glucolipid | | | 2.6 |
| C10-16 APG, 50 | | 10 | 10 |
| NaCl | 1.25 | | |
| Xanthan Gum | | 0.3 | 0.3 |
| Glycerin | | 2 | 2 |
| Rewopol ® SB CS 50 | | | 5 |
| Desferrioxamine | 0.1 | 0.1 | 0.1 |
| Preservative | q.s. | q.s. | q.s. |
| pH value | 5.5 | 5.5 | 5.5 |

Recipes 77, 78, 79: Cleaner for Plastic furniture and interieur

| Ingredient | 77 | 78 | 79 |
|---|---|---|---|
| Water | ad. 100 | ad. 100 | ad. 100 |
| Rhamnolipid | 0.5 | | |
| Sophorolipid | | 0.5 | |
| Glucolipid | | | 1.0 |
| Tego ® Betain C 60 | 1.5 | | |
| Tomakleen 91-6 | 1.5 | | |
| Rewoquat ® Q 70 | 0.5 | | |
| Rewopol ® CC 40 B MB | | 4 | |
| Tegotens ® DO 40 | | | 3.5 |
| Tegopren ® 6922 | | 1.2 | 1.2 |
| Desferrioxamine | 0.1 | 0.1 | 0.1 |
| pH value (adjust to) | 9 | 9 | 9 |

Recipes 80, 81, 82: Grill and Oven Cleaner

| Ingredient | 80 | 81 | 82 |
|---|---|---|---|
| Water | ad. 100 | ad. 100 | ad. 100 |
| MGDA | 5 | 5 | 5 |
| Sodium Carbonate | 5 | 5 | 5 |
| Rewoteric ® AM KSF 40 MB | 5 | | |
| Tomadol 91-6 | 1.5 | 1.5 | |
| Rhamnolipid | 0.75 | | |
| Sophorolipid | | 0.75 | |
| Glucolipid | | | 1.5 |
| Desferrioxamine | 1 | 1 | 1 |
| Dowanol DPnB | 1 | 1 | 1 |
| Rewoquat ® CQ 200 | | 5 | 5 |
| Tomakleen G-14 | | | 6 |
| pH (adjust to) | 12 | 12 | 12 |

Recipes 83, 84, 85: Cleaner for Food&Beverage Fatsolve

| Ingredient | 83 | 84 | 85 |
|---|---|---|---|
| Water | ad. 100 | ad. 100 | ad. 100 |
| Rewoquat ® CQ 200 | 20 | 12 | 12 |
| Rewoteric ® AM KSF 40 MB | 10 | | |
| Rhamnolipid | 2.5 | 2.5 | 1.5 |
| MGDA | 5 | | 5 |
| Sodium Carbonate | 3 | | 3 |
| Tomadol 91-8 | | 5 | |
| Tomadol 1-5 | | 3 | 2 |
| Desferrioxamine | 0.05 | 0.05 | 0.05 |
| pH value (adjust to) | 12 | 12 | 12 |

Recipes 86, 87, 88: Car Shampoo

| Ingredient | 86 | 87 | 88 |
|---|---|---|---|
| Water | ad. 100 | ad. 100 | ad. 100 |
| Tomakleen G-14 | 5 | 5 | |
| Rewoteric ® AM KSF 40 MB | 5 | | |
| MGDA | 3 | 2 | 3 |
| Sodium Carbonate | 1 | 2 | |
| Sodium laurylethersulfate, 28 | 10 | | |
| Rhamnolipid | | 0.25 | 1.5 |
| Sophorolipid | 1.5 | 1.5 | 1.5 |
| Tomadol 91-6 | | 3 | |
| Rewocare ® 755 | | 0.5 | 0.5 |
| Tego ® Betain C 60 | | 2 | 3 |
| Desferrioxamine | 0.25 | 0.25 | 0.25 |
| pH value (adjust to) | 9 | 10 | 9 |

Recipes 89, 90, 91: Car Rinse

| Ingredient | 89 | 90 | 91 |
|---|---|---|---|
| Water | ad. 100 | ad. 100 | ad. 100 |
| Carspray 90 MB | 20 | — | — |
| Rewopol ® MPG 40 | — | 5 | 5 |
| Sophorolipid | 0.25 | 0.25 | 0.5 |
| Rewoquat ® CR 3099 | — | 15 | — |
| Tegopren ® 6922 | — | — | 20 |
| Isopropanol | 2 | 2 | 2 |
| Desferrioxamine | 0.1 | 0.1 | 0.1 |
| pH value (adjust to) | 8 | 8 | 8 |

Recipes 92a, 92b, Automatic Dish Wash Powder

| Ingredient | 92a | 92b |
|---|---|---|
| Sodium carbonate | 42.0 | 42.0 |
| Sodium sulphate | 20.0 | 20.0 |
| Sodium citrate | 15.0 | 15.0 |
| Sodium percarbonate | 10.0 | 10.0 |
| Polymer | 3.0 | 3.0 |
| Phosphonate (HEDP) | 2.5 | 2.5 |
| Sodium metasilicate | 2.0 | 2.0 |
| Tomakleen G-14 | 1.0 | 1.0 |
| Sophorolipid | 0.5 | 0.5 |
| TAED | 2.0 | 2.0 |
| Protease | 1.0 | — |
| Amylase | 0.5 | — |
| Desferrioxamine | 0.2 | 0.2 |

Recipes 93a, 93b, 93c: Automatic Dish Wash Gel

| Ingredient | 93a | 93b | 93c |
|---|---|---|---|
| Water | 65 | 65 | 65 |
| Tetrasodium Glutamare diacetate / GLDA | 14 | 14 | 14 |
| Glycerol | 9.0 | 9.0 | 9.0 |
| Polyacrylate polymer | 2.0 | 2.0 | 2.0 |
| Protease | 1.0 | 1.0 | 1.0 |
| Formic acid | 0.2 | 0.2 | 0.2 |
| PPG-1 C6-10 Pareth-20 | 2.0 | 2.0 | 2.0 |
| Desferrioxamine | 2.0 | 2.0 | 2.0 |
| Amylase | 0.50 | 0.50 | 0.50 |
| Rhamnolipid | 0.25 | — | — |
| Sophorolipid | — | 0.25 | 0.1 |
| Glucolipid | — | — | 0.3 |
| Xanthan Gum | 0.50 | 0.50 | 0.50 |
| Perfumes | 0.20 | 0.10 | 0.10 |
| Zinc acetate dihydrate | 0.10 | 0.10 | 0.10 |
| Calcium Chloride | 0.10 | 0.050 | 0.050 |
| Limonen | 0.050 | 0.050 | 0.050 |
| Colorant | 0.050 | 0.050 | 0.050 |
| CIT/MIT | 0.050 | 0.050 | 0.050 |

Recipes 94a, 94b, 95a, 95b, 96, 97: Laundry pre-spotter

| Ingredient | 94 | 94b | 95a | 95b | 96 | 97 |
|---|---|---|---|---|---|---|
| Rhamnolipids | 2.5 | 2.5 | 6 | 6 | 2.5 | 2.5 |
| Sophorolipids | 6 | 6 | 2.5 | 2.5 | 0.5 | 0.5 |
| Lauryl/ | | | | | 12 | 12 |
| Myristil | | | | | | |
| Glucoside | | | | | | |
| Propylene | 2 | 2 | 2 | 2 | 2 | 2 |
| Glycol | | | | | | |
| Na citrate | 2 | 2 | 2 | 2 | 2 | 2 |
| Na formate | 0.5 | 0.5 | 0.5 | 0.5 | 0.5 | 0.5 |
| CaCl2 | 0.1 | 0.1 | 0.1 | 0.1 | 0.1 | 0.1 |
| NaOH | 0.05 | 0.05 | 0.05 | 0.05 | 0.05 | 0.05 |
| Protease | 1 | | 1 | | | 0.4 |
| Amylase | 0.4 | 0.4 | 0.4 | 0.4 | | 0.2 |
| Mannanase | 0.2 | 0.2 | 0.2 | 0.2 | | 0.2 |
| Pectinase | 0.2 | 0.2 | 0.2 | 0.2 | | 0.2 |
| Cellulase | 0.2 | 0.2 | 0.2 | 0.2 | | 0.2 |
| Desferriox- | 0.1% | 0.1% | 0.1% | 0.1% | 0.1% | 0.1% |
| amine | | | | | | |
| Water | ad 100 | ad 100 | ad 100 | ad 100 | ad 100 | ad 100 |

Recipes 98a, 98b, 98c, 99a, 99b, 99c: Laundry pre-spotter

| Ingredient | 98a | 98b | 98c | 99a | 99b | 99c |
|---|---|---|---|---|---|---|
| Rhamnolipids | 2.5 | 2.5 | — | 1.5 | 1.5 | — |
| Sophorolipids | — | — | 1.25 | — | — | 0.5 |
| Glucolipids | — | — | 2.5 | — | — | 2.0 |
| Lauryl/ | 12 | 12 | 12 | 9 | 9 | 9 |
| Myristil | | | | | | |
| Glucoside | | | | | | |
| Propylene | 2 | 2 | 2 | 2 | 2 | 2 |
| Glycol | | | | | | |
| Na citrate | 2 | 2 | 2 | 2 | 2 | 2 |
| Na formate | 0.5 | 0.5 | 0.5 | 0.5 | 0.5 | 0.5 |
| CaCl2 | 0.1 | 0.1 | 0.1 | 0.1 | 0.1 | 0.1 |
| NaOH | 0.05 | 0.05 | 0.05 | 0.05 | 0.05 | 0.05 |
| Protease | 1 | — | — | 1 | — | — |
| Amylase | 0.4 | — | — | 0.4 | — | — |
| Mannanase | 0.2 | — | — | 0.2 | — | — |
| Pectinase | 0.2 | — | — | 0.2 | — | — |
| Cellulase | 0.2 | — | — | 0.2 | — | — |
| Desferriox- | 0.2% | 0.2% | 0.2% | 0.2% | 0.2% | 0.2% |
| amine | | | | | | |
| Water | ad 100 | ad 100 | ad 100 | ad 100 | ad 100 | ad 100 |

Recipes 100a, 100b, 100c, 100d, 101a, 101b, 101c, 101d: Laundry pre-spotter

| Ingredient | 100a | 100b | 100c | 100d | 101a | 101b | 101c | 101d |
|---|---|---|---|---|---|---|---|---|
| Rhamnolipids | 2.5 | 2.5 | | | 0.5 | 0.5 | | |
| Sophorolipid | | | 1 | | | | 0.25 | |
| Glucolipid | | | 3 | 5 | | | 0.5 | 1.0 |
| Propylene Glycol | 2 | 2 | 2 | 2 | 2 | 2 | 2 | 2 |
| Na citrate | 2 | 2 | 2 | 2 | 2 | 2 | 2 | 2 |
| Na formate | 0.5 | 0.5 | 0.5 | 0.5 | 0.5 | 0.5 | 0.5 | 0.5 |
| CaCl2 | 0.1 | 0.1 | 0.1 | 0.1 | 0.1 | 0.1 | 0.1 | 0.1 |
| NaOH | 0.05 | 0.05 | 0.05 | 0.05 | 0.05 | 0.05 | 0.05 | 0.05 |
| Protease | 1 | 1 | 1 | 1 | 1 | 1 | 1 | 1 |
| Amylase | 0.4 | 0.4 | 0.4 | 0.4 | 0.4 | 0.4 | 0.4 | 0.4 |
| Mannanase | 0.2 | 0.2 | 0.2 | 0.2 | 0.2 | 0.2 | 0.2 | 0.2 |
| Pectinase | 0.2 | 0.2 | 0.2 | 0.2 | 0.2 | 0.2 | 0.2 | 0.2 |
| Cellulase | 0.2 | 0.2 | 0.2 | 0.2 | 0.2 | 0.2 | 0.2 | 0.2 |
| Water | ad 100 | ad 100 | ad 100 | ad 100 | ad 100 | ad 100 | ad 100 | ad 100 |

Recipes 102a, 102b, 102c, 102d, 102e, 102f: laundry detergents

| Ingredient | 102a | 102b | 102c | 102d | 102e | 102f |
|---|---|---|---|---|---|---|
| Rhamnolipids | 7.00 | 3.5 | 3.5 | 3.5 | 3.5 | 3.5 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | .0 |
| Linear alkylbenzene Sulfonates (LAS) | — | — | 0.5 | 5 | — | — |
| C8C16 Alkyl polyglucosid (Glucopon ® | — | — | — | — | 2.5 | — |
| 650 EC) | | | | | | |
| Amine oxide | — | — | — | 1 | — | — |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.00 | 5.00 | 5.00 | 5.00 | 5.00 | 5.00 |
| Amide polyglycol ether (Amidet ® N) | — | — | — | — | — | 1.0 |
| Polyethyleneimine PEI, 20 EO | 1.0 | 1.0 | 1.0 | 1.0 | 1.0 | 1.0 |
| (Sokalan ® HP20) | | | | | | |
| Sodium citrate | 3.50 | 3.50 | 3.50 | 3.50 | 3.50 | 3.50 |
| Sodium Phosphonate (Dequest ® | 2.0 | 2.0 | 2.0 | 2.0 | 2.0 | 2.0 |
| 2066) | | | | | | |
| Ethanol | 2.50 | 2.50 | 2.50 | 2.50 | 2.50 | 2.50 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.1 | 0.1 | 0.1 | 0.1 | 0.1 | 0.1 |
| Water | add 100 | add 100 | add 100 | add 100 | add 100 | add 100 |
| pH | 8.4 | 8.4 | 8.4 | 8.4 | 8.4 | 8.4 |

Recipes 102g, 102h, 102i, 102j, 102k, 102l: laundry detergents

| Ingredient | 102g | 102h | 102i | 102j | 102k | 102l |
|---|---|---|---|---|---|---|
| Sophorolipids | 7.00 | 3.5 | 3.5 | | 3.5 | |
| Glucolipids | | 7.00 | | 7.0 | | 7.0 |
| C12/14 7EO (Dehydol ® LT7) | — | — | 5.0 | 5.0 | 5.0 | 5.0 |
| SLES 2EO (Texapon ® N70) | — | — | 7.0 | 7.0 | 5.0 | 5.0 |
| Linear alkylbenzene Sulfonates (LAS) | — | — | — | — | 0.5 | 0.5 |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.00 | 5.00 | 5.00 | 5.00 | 5.00 | 5.00 |
| Polyethyleneimine PEI, 20 EO | 1.0 | 1.0 | 1.0 | 1.0 | 1.0 | 1.0 |
| (Sokalan ® HP20) | | | | | | |
| Sodium citrate | 3.50 | 3.50 | 3.50 | 3.50 | 3.50 | 3.50 |
| Sodium Phosphonate (Dequest ® 2066) | 2.0 | 2.0 | 2.0 | 2.0 | 2.0 | 2.0 |
| Ethanol | 2.50 | 2.50 | 2.50 | 2.50 | 2.50 | 2.50 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.1 | 0.1 | 0.1 | 0.1 | 0.1 | 0.1 |
| Water | add 100 | add 100 | add 100 | add 100 | add 100 | add 100 |
| pH | 8.4 | 8.4 | 8.4 | 8.4 | 8.4 | 8.4 |

Recipes 103a, 103b, 103c, 103d, 103e, 103f: laundry detergents

| Ingredient | 103a | 103b | 103c | 103d | 103e | 103f |
|---|---|---|---|---|---|---|
| Rhamnolipids | 7.0 | 3.5 | 3.5 | 3.5 | 3.5 | 3.5 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | 2.5 | 2.5 | — | — |
| C8C16 Alkyl polyglucosid (Glucopon ® 650 | — | — | — | — | 2.5 | — |
| EC) | | | | | | |
| Amine oxide | — | — | — | 1 | — | — |
| Sodium xylene sulfonate (Eltesol ® SX40) | 1.0 | 1.0 | 1.0 | 1.0 | 1.0 | 1.0 |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Vinylpyrrolidone/Vinylimidazole copolymer | 2.0 | 2.0 | 2.0 | 2.0 | 2.0 | 2.0 |
| (Sokalan ® HP 56) | | | | | | |
| Sodium citrate | 3.5 | 3.5 | 3.5 | 3.5 | 3.5 | 3.5 |
| Sodium Phosphonate (Dequest ® 2066) | 2.0 | 2.0 | 2.0 | 2.0 | 2.0 | 2.0 |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.2 | 0.2 | 0.2 | 0.2 | 0.2 | 0.2 |
| Water | add 100 | add 100 | add 100 | add 100 | add 100 | add 100 |
| pH | 8.4 | 8.4 | 8.4 | 8.4 | 8.4 | 8.4 |

Recipes 103g, 103h, 103i, 103j, 103k: laundry detergents

| Ingredient | 103g | 103h | 103i | 103j | 103k |
|---|---|---|---|---|---|
| Sophorolipids | 3.5 | 3.5 | — | 3.5 | — |
| Glucolipids | 7.0 | — | 7.0 | — | 7.0 |
| C12/14 7EO (Dehydol ® LT7) | — | 5.0 | 5.0 | 5.0 | 5.0 |
| SLES 2EO (Texapon ® N70) | — | 7.0 | 7.0 | 5.0 | 5.0 |
| Linear alkylbenzene Sulfonates (LAS) | — | — | — | 5 | 5 |
| Sodium xylene sulfonate (Eltesol ® SX40) | 1.0 | 1.0 | 1.0 | 1.0 | 1.0 |
| Na soap from palm kernel oil | 5.0 | 5.0 | 5.0 | 5.0 | 5.0 |
| Vinylpyrrolidone/Vinylimidazole copolymer | 2.0 | 2.0 | 2.0 | 2.0 | 2.0 |
| (Sokalan ® HP 56) | | | | | |
| Sodium citrate | 3.5 | 3.5 | 3.5 | 3.5 | 3.5 |
| Sodium Phosphonate (Dequest ® 2066) | 2.0 | 2.0 | 2.0 | 2.0 | 2.0 |
| Ethanol | 2.5 | 2.5 | 2.5 | 2.5 | 2.5 |
| MPG | 1.0 | 1.0 | 1.0 | 1.0 | 1.0 |
| Boric acid | 1.0 | 1.0 | 1.0 | 1.0 | 1.0 |
| Phenoxyethanol (Acticide ® SR150) | 0.45 | 0.45 | 0.45 | 0.45 | 0.45 |
| Liquanase ® 3.5 L | 1.0 | 1.0 | 1.0 | 1.0 | 1.0 |
| Amplify ® Prime 100 L | 0.4 | 0.4 | 0.4 | 0.4 | 0.4 |
| Mannaway ® 200 L | 0.2 | 0.2 | 0.2 | 0.2 | 0.2 |
| Carezyme ® Premium 4500 L | 0.2 | 0.2 | 0.2 | 0.2 | 0.2 |
| XPect ® 1000 L | 0.2 | 0.2 | 0.2 | 0.2 | 0.2 |
| Celluclean ® 5000 L | 0.2 | 0.2 | 0.2 | 0.2 | 0.2 |
| Pristine ® 100 L | 0.1 | 0.1 | 0.1 | 0.1 | 0.1 |
| NaOH | 1.0 | 1.0 | 1.0 | 1.0 | 1.0 |
| Desferrioxamine | 0.2 | 0.2 | 0.2 | 0.2 | 0.2 |
| Water | add 100 | add 100 | add 100 | add 100 | add 100 |
| pH | 8.4 | 8.4 | 8.4 | 8.4 | 8.4 |

Recipes 104a, 104b, 104c, 104d, 104e, 104f: laundry detergents

| Ingredient | 104a | 104b | 104c | 104d | 104e | 104f |
|---|---|---|---|---|---|---|
| Rhamnolipids | 7.0 | 3.5 | 3.5 | 3.5 | 3.5 | 3.5 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | .5 | 5 | — | — |
| C8C16 Alkyl polyglucosid (Glucopon ® | — | — | — | — | 2.5 | — |
| 650 EC) | | | | | | |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Texcare ® SRN 170 | 2.0 | 2.0 | 2.0 | 2.0 | 2.0 | 2.0 |
| Sodium citrate | 3.5 | 3.5 | 3.5 | 3.5 | 3.5 | 3.5 |
| Sodium Phosphonate (Dequest ® | 2.0 | 2.0 | 2.0 | 2.0 | 2.0 | 2.0 |
| 2066) | | | | | | |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.2 | 0.2 | 0.2 | 0.2 | 0.2 | 0.2 |
| Water | add 100 | add 100 | add 100 | add 100 | add 100 | add 100 |
| pH | 8.4 | 8.4 | 8.4 | 8.4 | 8.4 | 8.4 |

Recipes 104g, 104h, 104i, 104j, 104k: laundry detergents

| Ingredient | 104g | 104h | 104i | 104j | 104k |
|---|---|---|---|---|---|
| Sophorolipids | 3.0 | 3.5 | — | 3.5 | — |
| Glucolipids | 8.0 | — | 7.0 | — | 7.0 |
| C12/14 7EO (Dehydol ® LT7) | — | 5.0 | 5.0 | 5.0 | 5.0 |
| SLES 2EO (Texapon ® N70) | — | 7.0 | 7.0 | 5.0 | 5.0 |
| Linear alkylbenzene Sulfonates | — | — | — | .5 | .5 |
| (LAS) | | | | | |
| C8C16 Alkyl polyglucosid | — | — | — | — | — |
| (Glucopon ® 650 EC) | | | | | |
| Sodium xylene sulfonate (Eltesol ® | 1.0 | 1.0 | 1.0 | 1.0 | 1.0 |
| SX40) | | | | | |
| Na soap from palm kernel oil | 5.0 | 5.0 | 5.0 | 5.0 | 5.0 |
| Amide polyglycol ether (Amidet ® N) | — | — | — | — | — |
| Texcare ® SRN 170 | 2.0 | 2.0 | 2.0 | 2.0 | 2.0 |
| Sodium citrate | 3.5 | 3.5 | 3.5 | 3.5 | 3.5 |
| Sodium Phosphonate (Dequest ® | 2.0 | 2.0 | 2.0 | 2.0 | 2.0 |
| 2066) | | | | | |
| Ethanol | 2.5 | 2.5 | 2.5 | 2.5 | 2.5 |
| MPG | 1.0 | 1.0 | 1.0 | 1.0 | 1.0 |
| Boric acid | 1.0 | 1.0 | 1.0 | 1.0 | 1.0 |
| Phenoxyethanol (Acticide ® SR150) | 0.45 | 0.45 | 0.45 | 0.45 | 0.45 |
| Liquanase ® 3.5 L | 1.0 | 1.0 | 1.0 | 1.0 | 1.0 |
| Amplify ® Prime 100 L | 0.4 | 0.4 | 0.4 | 0.4 | 0.4 |
| Mannaway ® 200 L | 0.2 | 0.2 | 0.2 | 0.2 | 0.2 |
| Carezyme ® Premium 4500 L | 0.2 | 0.2 | 0.2 | 0.2 | 0.2 |
| XPect ® 1000 L | 0.2 | 0.2 | 0.2 | 0.2 | 0.2 |
| Celluclean ® 5000 L | 0.2 | 0.2 | 0.2 | 0.2 | 0.2 |
| Pristine ® 100 L | 0.1 | 0.1 | 0.1 | 0.1 | 0.1 |
| NaOH | 1.0 | 1.0 | 1.0 | 1.0 | 1.0 |
| Desferrioxamine | 0.2 | 0.2 | 0.2 | 0.2 | 0.2 |
| Water | add 100 | add 100 | add 100 | add 100 | add 100 |
| pH | 8.4 | 8.4 | 8.4 | 8.4 | 8.4 |

Recipes 105a, 105b, 105c, 105d, 105e, 105f: laundry detergents

| Ingredient | 105a | 105b | 105c | 105d | 105e | 105f |
|---|---|---|---|---|---|---|
| Rhamnolipids | 7.0 | 3.5 | 3.5 | 3.5 | 3.5 | 3.5 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | .5 | 5 | — | — |
| C8C16 Alkyl polyglucosid (Glucopon ® | — | — | — | — | 2.5 | — |
| 650 EC) | | | | | | |
| Amine oxide | — | — | — | 1 | — | — |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Texcare ® SRN 170 | 2.0 | 2.0 | 2.0 | 2.0 | 2.0 | 2.0 |
| Sodium citrate | 3.5 | 3.5 | 3.5 | 3.5 | 3.5 | 3.5 |
| Sodium Phosphonate (Dequest ® | 2.0 | 2.0 | 2.0 | 2.0 | 2.0 | 2.0 |
| 2066) | | | | | | |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.1 | 0.1 | 0.1 | 0.1 | 0.1 | 0.1 |
| Water | add 100 | add 100 | add 100 | add 100 | add 100 | add 100 |
| pH | 8.4 | 8.4 | 8.4 | 8.4 | 8.4 | 8.4 |

Recipes 105g, 105h, 105i, 105j, 105k, 105l: laundry detergents

| Ingredient | 105g | 105h | 105i | 105j | 105k | 105l |
|---|---|---|---|---|---|---|
| Glucolipids | 12.0 | 6.0 | 6.0 | 6.0 | 6.0 | 6.0 |
| Sophorolipids | 1.0 | 0.5 | 0.5 | 0.5 | 0.5 | 0.5 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | .5 | 5 | — | — |
| C8C16 Alkyl polyglucosid (Glucopon ® | — | — | — | — | 2.5 | — |
| 650 EC) | | | | | | |
| Amine oxide | — | — | — | 1 | — | — |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Texcare ® SRN 170 | 2.0 | 2.0 | 2.0 | 2.0 | 2.0 | 2.0 |
| Sodium citrate | 3.50 | 3.50 | 3.50 | 3.50 | 3.50 | 3.50 |
| Sodium Phosphonate (Dequest ® | 2.0 | 2.0 | 2.0 | 2.0 | 2.0 | 2.0 |
| 2066) | | | | | | |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 0.1 | 0.1 | 0.1 | 0.1 | 0.1 | 0.1 |
| Water | add 100 | add 100 | add 100 | add 100 | add 100 | add 100 |
| pH | 8.4 | 8.4 | 8.4 | 8.4 | 8.4 | 8.4 |

Recipes 106a, 106b, 106c, 106d, 106e, 106f: laundry detergents

| Ingredient | 106a | 106b | 106c | 106d | 106e | 106f |
|---|---|---|---|---|---|---|
| Rhamnolipids | 7.00 | 3.5 | 3.5 | 3.5 | 3.5 | 3.5 |
| C12/14 7EO (Dehydol ® LT7) | — | 5.0 | 5.0 | 2.5 | | 5.0 |
| SLES 2EO (Texapon ® N70) | — | 7.0 | 5.0 | 2.5 | 5.0 | 5.0 |
| Linear alkylbenzene Sulfonates | — | — | 0.5 | 5 | — | — |
| (LAS) | | | | | | |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.00 | 5.00 | 5.00 | 5.00 | 5.00 | 5.00 |
| Texcare ® SRN 170 | 1 | 1 | 1 | 1 | 1 | — |
| Sodium citrate | 6 | 6 | 6 | 6 | 6 | 6 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 1.0 | 1.0 | 1.0 | 1.0 | 1.0 | 1.0 |
| Water | add 100 | add 100 | add 100 | add 100 | add 100 | add 100 |
| pH | 8.4 | 8.4 | 8.4 | 8.4 | 8.4 | 8.4 |

Recipes 106g, 106h, 106i, 106j, 106k, 106l: laundry detergents

| Ingredient | 106g | 106h | 106i | 106j | 106k | 106l |
|---|---|---|---|---|---|---|
| Glucolipids | 14.00 | | 7.0 | | 7.0 | |
| Sophorolipids | — | 7.00 | — | 3.5 | — | 3.5 |
| C12/14 7EO (Dehydol ® LT7) | — | — | 5.0 | 5.0 | 5.0 | 5.0 |
| SLES 2EO (Texapon ® N70) | — | — | 7.0 | 7.0 | 5.0 | 5.0 |
| Linear alkylbenzene Sulfonates | — | — | — | — | 0.5 | 0.5 |
| (LAS) | | | | | | |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.00 | 5.00 | 5.00 | 5.00 | 5.00 | 5.00 |
| Texcare ® SRN 170 | 1 | 1 | 1 | 1 | 1 | 1 |
| Sodium citrate | 6 | 6 | 6 | 6 | 6 | 6 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 1.0 | 1.0 | 1.0 | 1.0 | 1.0 | 1.0 |
| Water | add 100 | add 100 | add 100 | add 100 | add 100 | add 100 |
| pH | 8.4 | 8.4 | 8.4 | 8.4 | 8.4 | 8.4 |

1. A composition comprising
A) at least one substance selected from the group consisting of a desferrioxamine and an acid addition product of a desferrioxamine or a salt thereof,
B) at least one biosurfactant, and optionally
C) at least one non-bio surfactant.

2. The composition according to claim 1, wherein said at least one substance is a desferrioxamine which is at least one selected from the group consisting of desferrioxamine A_{1A}, desferrioxamine A_{1B}, desferrioxamine A₂, desferrioxamine B, desferrioxamine D₁, desferrioxamine D₂, desferrioxamine E, desferrioxamine Et₁, desferrioxamine Et₂, desferrioxamine Et₃, desferrioxamine G₁, desferrioxamine G_{2A}, desferrioxamine G_{2B}, desferrioxamine G_{2C}, desferrioxamine H, desferrioxamine N, desferrioxamine P₁, desferrioxamine T₁, desferrioxamine T₂, desferrioxamine T₃, desferrioxamine T₇, desferrioxamine T₈, desferrioxamine Te₁, desferrioxamine Te₂, desferrioxamine Te₃, desferrioxamine X₁, desferrioxamine X₂, desferrioxamine X₃, and desferrioxamine X₄.

3. The composition according to claim 1, wherein said at least one substance is an acid addition product of a desferrioxamine or a salt thereof which is at least one selected from the group consisting of formic acid, aminooxyacetic acid, an amino acid, anthraquinonecarboxylic acid, succinic acid, aqueous hydrochloric acid, acetic acid, folic acid, glutaric acid, hydroxamic acid, malonic acid, methanesulfonic acid, nalidixic acid, N-(methylamino)benzoic acid, phenylsuccinic acid, phenylglutaric acid, phosphorous acid, phosphoric acid, sulfurous acid, sulfuric acid, tartaric acid, and citric acid.

4. The composition according to claim 1, wherein the biosurfactant is at least one selected from the group consisting of a rhamnolipid, a glucolipid, and a sophorolipid.

5. The composition according to claim 1, wherein the composition comprises the component A) in an amount from 0.10% by weight to 75.0% by weight, based on the total composition.

6. The composition according to claim 1, wherein the composition comprises the component B) in an amount from 5.0% by weight to 80% by weight, based on the total composition.

7. The composition according to claim 1, wherein the composition comprises the component C) in an amount from 1.0% by weight to 50% by weight, based on the total composition.

8. The composition according to claim 1, wherein the non-bio surfactant is at least one selected from the group consisting of an anionic, a cationic, a non-ionic, a semi-polar, and a zwitterionic surfactant.

9. The composition according to claim 1, wherein a sum total of all surfactants is from 5.0% by weight to 90% by weight, wherein the percentages by weight relate to the total composition.

10. The composition according to claim 1, wherein a sum of biosurfactant in the total surfactant is from 1% by weight to 90% by weight.

11. The composition according to claim 1 further comprising
water.

12. The composition according to claim 1, wherein the composition is at least one formulation selected from the group consisting of a cosmetic formulation, a laundry formulation, and a dishwashing formulation.

13. The composition according to claim 12, wherein the composition comprises the component A) in an amount from 0.005% by weight to 10.0% by weight, based on the total composition.

14. The composition according to claim 12, wherein the composition comprises the component B) in an amount from 0.05% by weight to 25.0% by weight, based on the total composition.

15. A method of removing dirt comprising applying to a surface the composition according to claim 1.
